(19) United States
(12) Patent Application Publication
(10) Pub. No.: US 2025/0152196 A1
(43) Pub. Date: May 15, 2025

(54) OPEN LOOP ÉCRASEUR FOR TENOTOMY

(71) Applicant: INCITE MEDICAL SARL, Geneva (CH)

(72) Inventors: Alexandre LÄDERMANN, Soral (CH); Frank Kolo CHRISTOPHE, Bernex (CH); Dominique PELLATON, Bernex (CH); Alexandre Ioan ROMOSCANU; Nichita MILODIN, Bucharest (RO); Stuart J. OLSTAD, Marcell, MN (US)

(21) Appl. No.: 18/839,290

(22) PCT Filed: Feb. 18, 2023
(86) PCT No.: PCT/US2023/013387

§ 371 Date: Aug. 16, 2024

Related U.S. Application Data
(60) Provisional application No. 63/311,580, filed on Feb. 18, 2022.

(51) Int. Cl. A61B 17/3205; A61B 17/32; A61B 17/34

(52) U.S. Cl. CPC A61B 17/32056; A61B 17/34; A61B 2017/32006

(57) ABSTRACT

An open loop percutaneous écraseur. The disclosed écraseur enable circumscription of elongate organs or tissue, such as tendons and blood vessels, which cannot be circumscribed by closed loop forms. The device is “open loop” because initially a cutting wire is routed around the cutting target. A variety of methods and structure for closing the écraseur loop are disclosed. The disclosed écraseur pierce and are anchored in the targeted tissue, thereby limiting relative motion of the écraseur relative to the cutting target. Severance of the target is controlled entirely from the proximal end of the instrument and performed centripetally, thereby avoiding risks associated with the use of cutting blade devices. The cutting target does not have to be accessed by open surgical techniques to complete the circumscription, enabling a minimally invasive severance with a single exterior incision that is smaller than the target to be cut.

### RELATED APPLICATIONS

This patent application claims the benefit of U.S. Provisional Patent Application No. 63/311,580, filed Feb. 18, 2022, the disclosure of which is incorporated by reference herein in its entirety.

### FIELD OF THE DISCLOSURE

This disclosure is directed generally to percutaneous devices and more specifically to a percutaneous écraseurs.

### BACKGROUND OF THE DISCLOSURE

Arthroscopically guided biceps tenotomy is a common outpatient surgical procedure performed by orthopedic surgeons today. Such procedures are usually performed under visual control with either disposable blade scalpels, the sharp edge of needles, electrocoagulation, electro frequency devices that provide soft tissue ablation (vaporization), lasers cutter that coagulates soft tissue, or for instance soft tissue ultrasonic cutter that comprised of aspirator, hemostatic blade and debridement blade. Such surgical interventions are usually performed under general anesthesia. However, surgical repairs are not always possible or refused by patients.

A promising alternative is percutaneous tenotomy of the long head of the biceps tendon with a cutting device. Presently, there are reliability concerns because of the high rate of partial section only, and iatrogenic injuries such as sectioning of adjacent structures and lacerations on the cartilage of the humeral head. Because of the inherent risks, a non-negligible number of patients do not wish to undergo shoulder arthroscopic surgery due to the concern for negative outcomes. There is subsequently a need for development of a reliable percutaneous tenotomic instrument.

### SUMMARY OF THE DISCLOSURE

Various embodiments of the disclosure present ultrasound-guided percutaneous écraseur for achieving tenotomy/soft tissue sections on an outpatient basis. The disclosed écraseurs perform tenotomy, for example of the long head of the biceps tendon, with local anesthesia. The procedure can be performed on ambulatory patients, without general anesthesia and with low morbidity.

The disclosed embodiments are directed to the cutting of tendons by way of example, but may also have other applications. More generally, the disclosed tenotomic écraseurs enables circumscription of elongate organs or tissue, such as tendons and blood vessels, which cannot be circumscribed by closed loop forms. Initially, leading and threading of a wire is performed by creating a deflecting and aligning path through and around the around the object to be cut (tendon or vessel). (Herein, “tenotomic” is defined as being of or relating to tenotomy, a surgical act which involves the division of a tendon.) As such, the disclosed écraseurs are referred to as “open loop,” for which various means for closing for a cutting loop are also disclosed. The procedures are controlled entirely from the proximal end of the instrument.

The disclosed écraseurs also prevent relative motion between the tendon and the device during the intervention, prevents any relative damage by limiting the insertion of sharp devices or tools inside the body, and secures a controlled cutting of the tendon. Severance of the tendon is performed centripetally provided by continuous shearing with a wire through the tissue, thereby avoiding the use of blades and other open cutting devices and the attendant risk to neighboring tissue and vessels. The tendon or vessel does not have to be accessed by open surgical techniques to complete the circumscription, enabling a minimally invasive severance with a single exterior incision that is smaller than required by open surgical techniques and with less disturbance to surrounding tissues.

Structurally, an open loop écraseur is presented with various embodiments for closure of the loop around a cutting target. A trocar defines and is collinear with a central axis, the trocar including a distal end portion that defines a sharp distal tip for percutaneous insertion, the distal end portion defining a first lateral opening proximate the sharp distal tip. A central stem is dimensioned for a sliding axial translation within the trocar, the central stem including a first lateral side for alignment adjacent the first lateral opening. A first steerable catheter is disposed in the trocar along the first lateral side of the central stem, the first steerable catheter defining a first working channel and including a distal steering section that is extendible radially outward through the first lateral openings, a distal extremity of the steering portion being steerable radially inward toward the first lateral opening. A cutting wire assembly at least partially disposed in the trocar. The first steerable catheter is retractable within the trocar in a proximal direction to expose the cutting wire assembly for contraction through the cutting target.

In some embodiments, at least one snare assembly that defines a snare axis and is axially translatable within an internal bypass defined within the trocar is used to close the écraseur loop. Each of the at least one snare assembly may include a snare portion at a distal end thereof for coupling with a first end portion of the cutting wire assembly, the first end portion being disposed in the first working channel of the first steerable catheter, the internal bypass being in fluid communication with the distal end portion of the trocar for translation of the snare portion into the distal end portion of the trocar. The end portion may be extendible through the first working channel to exit the distal extremity of the distal steering section of the first steerable catheter for coupling with the snare portion, the snare assembly being retractable in a proximal direction for the at least partial circumscription of the cutting target.

In some embodiments, a first snare assembly is translatable within the first working channel of the first steerable catheter and includes a first snare portion at a distal end thereof for coupling with a first end portion of the cutting wire assembly, he first snare portion being extendible through the first working channel to exit a distal extremity of the distal steering section of the first steerable catheter for positioning the snare portion adjacent and exterior to the first lateral opening of the trocar. A first exit port may be defined at a distal end of the central stem, the first exit port being in fluid communication with an internal bypass defined within the trocar that extends axially and proximally from the first exit port, the first exit port being in fluid communication with the distal end portion of the trocar. In some embodiments, the first exit port defines an exit port axis that extends radially outward in a distal direction for deflection of the first end portion through the first lateral opening of the trocar for coupling with the snare portion, the first end portion of the cutting wire assembly being disposed within the internal bypass and extending through the first exit port. The first snare assembly and the central stem may be retractable in a proximal direction for the at least partial circumscription of the cutting target.

In some embodiments, closure of the open loop écraseur is performed with a first snare assembly that is translatable within the first working channel of the first steerable catheter that includes a first snare portion at a distal end thereof for coupling with a first end portion of the cutting wire assembly. The first snare portion may be extendible through the first working channel to exit a distal extremity of the distal steering section of the first steerable catheter for positioning the snare portion adjacent and exterior to the first lateral opening of the trocar. In some embodiments, an internal bypass defined within the trocar extends axially and is in fluid communication with the distal end portion of the trocar, the first end portion being disposed within the internal bypass and extending into the distal end portion of the trocar and around and proximal to a distal end of the central stem to extend axially and along the first lateral side of the central stem and adjacent the first lateral opening. In some embodiments, translation of the first end portion of the cutting wire assembly in a proximal direction within the internal bypass causes a distal extremity of the first end portion of the cutting wire assembly to deflect radially outward through the first lateral opening for coupling with the first snare portion.

In some embodiments, a snare assembly is translatable within the first working channel of the first steerable catheter and includes a snare portion at a distal end thereof for coupling with a first end portion of the cutting wire assembly, the snare portion being extendible through the first working channel to exit a distal extremity of the distal steering section of the first steerable catheter for positioning the snare portion adjacent and exterior to the first lateral opening of the trocar. A second steerable catheter may be disposed in the trocar along a second lateral side of the central stem, the second steerable catheter defining a second working channel and including a distal steering section that is extendible radially outward through a second lateral opening defined at the distal end portion of the trocar proximate the sharp distal tip, a distal extremity of the second steerable catheter being steerable radially inward toward the second lateral opening. In some embodiments, the first end portion of the cutting wire assembly is disposed in the second working channel of the second steerable catheter, a distal extremity of the first end portion being routed through the first lateral opening and the second lateral opening for coupling with the snare portion. The snare portion may be coupled to the first end portion of the cutting wire assembly and the snare assembly is retracted to draw the cutting wire assembly into the first working channel for the at least partial circumscription of the cutting target.

In some embodiments, the open loop of the écraseur is closed using a first end portion of the cutting wire assembly disposed in the first working channel of the first steerable catheter, and a second steerable catheter disposed in the trocar along a second lateral side of the central stem, the second steerable catheter defining a second working channel and including a distal steering section that is extendible radially outward through a second lateral opening defined at the distal end portion of the trocar proximate the sharp distal tip. A distal extremity of the second steerable catheter may be steerable radially inward toward the second lateral opening. In some embodiments, the distal steering section of the first steerable catheter is steerable through the first lateral opening and the distal steering section of the second steerable catheter is steerable through the second lateral opening for lateral alignment of the distal extremities of the first steerable catheter and the second steerable catheter within the distal end portion of the trocar. The first end portion of the cutting wire assembly may be extendible from the first working channel into the second working channel to at least partially circumscribe the cutting target with the cutting wire assembly.

### BRIEF DESCRIPTION OF THE DRAWINGS

FIG. 1 is a perspective view of an open loop écraseur in a tenotomy procedure according to an embodiment of the disclosure;

FIGS. 2 through 5 are schematic views depicting an open loop écraseur for effectuating a bifurcated cutting loop configuration according to an embodiment of the disclosure;

FIGS. 6 and 7 are schematic views depicting an open loop écraseur for effectuating a symmetrical cutting loop configuration according to an embodiment of the disclosure;

FIGS. 8 and 9 are schematic views depicting an open loop écraseur for effectuating an asymmetrical cutting loop configuration according to an embodiment of the disclosure;

FIG. 10 is a partial sectional and cutaway view of an open loop écraseur according to a first embodiment of the disclosure;

FIG. 11 is a partial perspective view of a central stem for the open loop écraseur of FIG. 10 according to an embodiment of the disclosure;

FIG. 12 is a sectional view of the open loop écraseur at cross-section XII-XII of FIG. 12 according to an embodiment of the disclosure;

FIG. 13 is a schematic representation of a cutting wire assembly according to an embodiment of the disclosure;

FIG. 14 is a perspective view of a slotted steering tube in assembly in an unactuated configuration according to an embodiment of the disclosure;

FIG. 15 is a sectional view of the slotted steering tube as assembled in FIG. 14 according to an embodiment of the disclosure;

FIG. 15A is a sectional view of the slotted steering tube of FIG. 14 in an alternative assembly according to an embodiment of the disclosure;

FIG. 16 is a sectional view of the slotted tube of FIG. 14 in isolation and in a flexed condition according to an embodiment of the disclosure;

FIG. 17 is a perspective view of a multi-lumen steering section according to an embodiment of the disclosure;

FIG. 18 is a sectional view of the multi-lumen steering section at plane XVIII of FIG. 17 according to an embodiment of the disclosure;

FIG. 19 is a perspective view of a single lumen steering section with a leaf spring disposed therein according to an embodiment of the disclosure;

FIG. 20 is a sectional view of the single lumen steering section at plane XX of FIG. 19 according to an embodiment of the disclosure;

FIGS. 21A through 21I depicts schematic views of an operating procedure for the open loop écraseur of FIG. 10 according to an embodiment of the disclosure;

FIG. 22 is s partial perspective view of an open loop écraseur according to a second embodiment of the disclosure;

FIG. 23 is a partial perspective view of a central stem of the open loop écraseur of FIG. 22 according to an embodiment of the disclosure;

FIG. 24 is a sectional view of the open loop écraseur at cross-section XXIV-XXIV of FIG. 22 according to an embodiment of the disclosure;

FIG. 25 is a partial perspective view of a snare assembly of the open loop écraseur of FIG. 22 according to an embodiment of the disclosure;

FIG. 26A through 26H depicts schematic views of an operating procedure for the open loop écraseur of FIG. 22 according to an embodiment of the disclosure;

FIG. 27 is a partial perspective view of an open loop écraseur in an insertion configuration according to a third embodiment of the disclosure;

FIG. 28 is a partial perspective view of the open loop écraseur of FIG. 27 in a deployed configuration according to an embodiment of the disclosure;

FIG. 29 is a sectional view of the open loop écraseur at cross-section 29-29 of FIG. 27 according to an embodiment of the disclosure;

FIG. 30 is a sectional view of the open loop écraseur at cross-section 30-30 of FIG. 27 according to an embodiment of the disclosure;

FIGS. 31A through 31H depicts schematic views of an operating procedure for the open loop écraseur of FIG. 27 according to an embodiment of the disclosure;

FIG. 32 is a partial perspective view of an open loop écraseur according to a fourth embodiment of the disclosure;

FIGS. 33A through 33H depicts schematic views of an operating procedure for the open loop écraseur of FIG. 32 according to an embodiment of the disclosure;

FIG. 34 is a partial perspective view of an open loop écraseur according to a fifth embodiment of the disclosure;

FIG. 35 is an exploded view of internal components of the open loop écraseur of FIG. 34 according to an embodiment of the disclosure;

FIG. 36 is an enlarged partial view of a stem portion of the open loop écraseur of FIG. 34 according to an embodiment of the disclosure;

FIG. 37 is a sectional view of the open loop écraseur at cross-section 37-37 of FIG. 34 according to an embodiment of the disclosure;

FIGS. 38A through 38H depicts schematic views of an operating procedure for the open loop écraseur of FIG. 34 according to an embodiment of the disclosure;

FIG. 39 is a partial perspective view of a central stem and distal alignment head in assembly for an open loop écraseur in a retracted configuration according to a sixth embodiment of the disclosure;

FIG. 40 is a partial perspective view of the central stem and distal alignment head of the open loop écraseur of FIG. 39 in an expanded configuration

FIG. 41 is a sectional view of the central stem at plane XLI of FIG. 39 in assembly in an open loop écraseur according to an embodiment of the disclosure;

FIG. 42 is a sectional view of the central stem at plane XLII of FIG. 40; and

FIG. 43A through 43H depicts schematic views of an operating procedure for the open loop écraseur of FIG. 41 according to an embodiment of the disclosure.

### DETAILED DESCRIPTION OF THE FIGURES

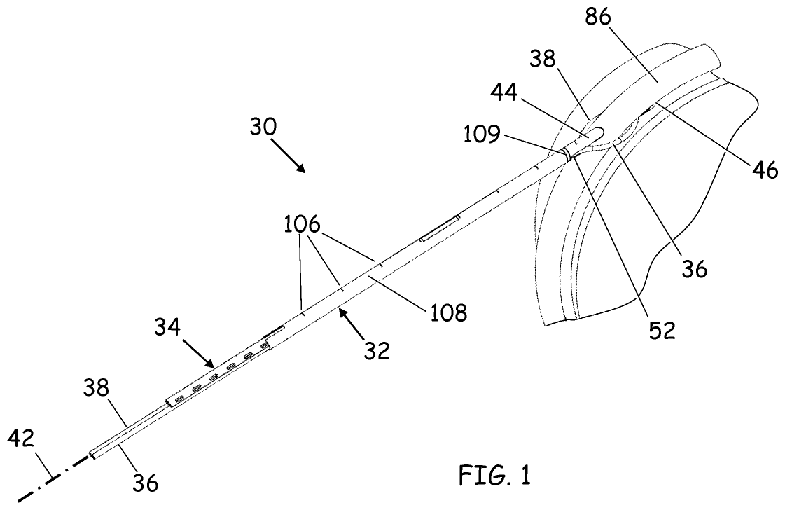

Referring to FIGS. 1 through 9, an open loop écraseur 30 is depicted according to embodiments of the disclosure. The open loop écraseur 30 includes an access tube 32 that houses a central stem 34 and a steerable catheter 36. Some embodiments include a second steerable catheter 38 (depicted). The open loop écraseur 30 also includes a cutting wire assembly 40 that is at least partially disposed in the access tube 32.

The access tube 32 defines and is collinear with a central axis 42 and includes a distal end portion 44 that defines a sharp distal tip 46 for percutaneous insertion. The distal end portion 44 also defines lateral openings 52 and 54 as egress for the steerable catheters 36 and 38, respectively. Because of structural similarities, the access tube is herein referred to as a trocar 32.

The central stem 34 is dimensioned for a sliding axial translation within the trocar 32 and includes a distal end 60 and first and second lateral sides 62 and 64 for alignment adjacent the lateral openings 52 and 54. The central stem 34 may also be rotatable within the trocar 32. In some embodiments, the central stem 34 includes catheter deflector portions 66 and 68, one for each of the steerable catheters 36 and 38, that are tangentially aligned with the lateral openings 52 and 54, for deflection of the steerable catheters 36 and 38 radially outward through the lateral openings 52 and 54. Alternatively, the steering function of the steerable catheters 36 and 38 may operate to initially cause the radial outward deflection through the lateral openings 52 and 54. The catheter deflector portions 66 and 68 each define a deflector surface 72 that extends radially outward relative to the central axis 42 and in a distal direction 74. In some embodiments, an internal bypass 80 is defined within the trocar 32

The internal bypass 80 is so-named because it provides internal access to the distal end portion 44 of the trocar 32 when the central stem 34 is in place other than other than through the lateral openings 52, 54. The internal bypass 80 defines a bypass axis 79 may be defined within the cross-section of the central stem 34 (depicted) or may be defined cooperatively between the central stem 34 and an interior surface 81 of the trocar 32.

The steerable catheters 36 and 38 are disposed in the trocar 32 along the first and second lateral sides 62 and 64, respectively, of the central stem 34. Each of the steerable catheters 36 and 38 define respective working channels 76 and may define respective steering wire channels 78 (e.g., FIG. 15) dedicated to the routing of steering wires. Each steerable catheter 36, 38 includes a distal steering section 82 that can be extended radially outward through the respective lateral openings 52 and 54. Each of the steerable catheters 36 and 38 are configured and arranged so that a distal extremity 84 can be steered radially inward toward the respective lateral openings 52 and 54.

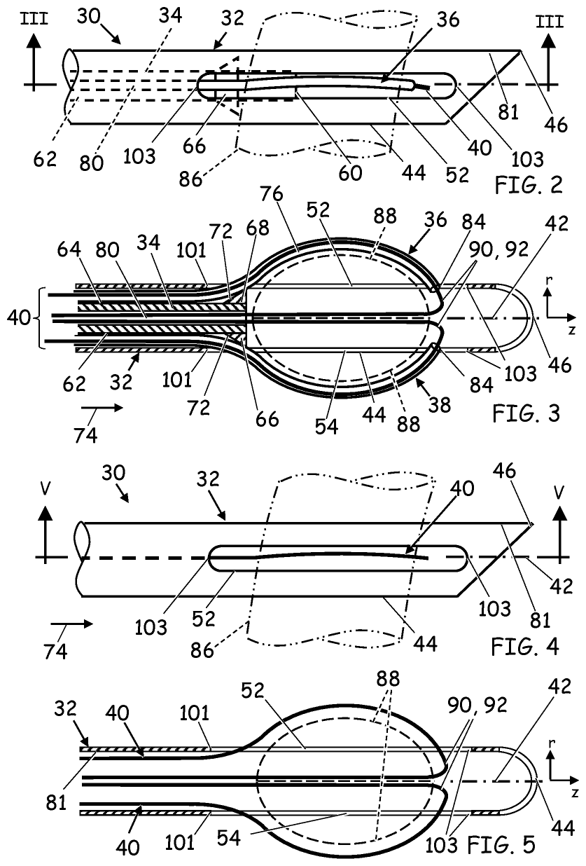

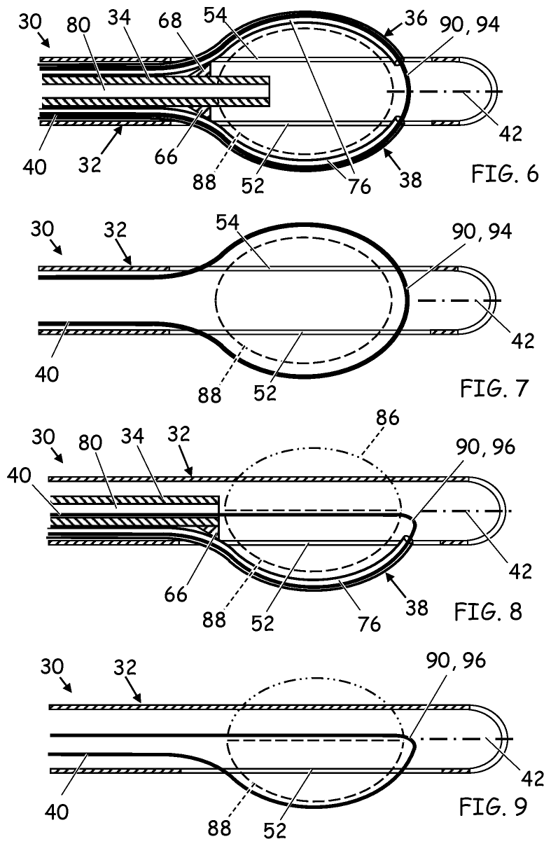

In operation, the trocar 32 is inserted through or adjacent a targeted organ or tissue 86 with the cutting wire assembly 40 in an open loop (free-ended) configuration. The cutting wire assembly 40 is circumscribed about the targeted organ or tissue 86 to form an écraseur loop 90 about a cutting target 88 (FIGS. 2 and 3). The écraseur loop 90 is then exposed to the targeted organ or tissue 86 (FIGS. 4 and 5) and contracted inward to cut through the cutting target 88. Severance may be assisted by reciprocal action of the cutting wire portion 156 (sawing) during contraction of the écraseur loop 90. Various configurations for the closed écraseur loop 90 are disclosed herein, including a bifurcated loop configuration 92 (FIGS. 3 and 5), a single symmetric loop configuration 94 (FIGS. 6 and 7), and a single asymmetric loop configuration 96.

For the various open loop écraseurs 30 disclosed herein, each of the respective lateral openings 52 and 54 may define a longitudinal axis 102 that pass through a proximal end 101 and a distal end 103 of the opening 52, 54 in a direction parallel to the central axis 42. In some embodiments, the longitudinal axes 102 are coplanar with a plane 104 that is radially offset from the central axis 42 (FIG. 11). Alternatively, the longitudinal axes 102 may be diametrically opposed about the central axis 42. Also, rather than single, elongate openings, a plurality of linearly arranged circular openings (not depicted) to provide egress and ingress along the distal end portion 44 of the trocar 32 is also contemplated. Other adequate shapes and arrangements may be implemented that define lateral egress and ingress for the steerable catheters 36 and 38. The trocar 32 may be metallic, and the sharp distal tip 46 may be beveled (depicted). The trocar 32 may include at least one marking 106 on an external surface 108 (e.g., by etching, slotting, scoring, indentation, or embossing) to aid in determining an insertion depth of the trocar 32. The at least one marking 106 may be a plurality of markings that spaced apart as gradations. In some embodiments, the trocar 32 includes one or more radiopaque marker bands 109, for example marking a proximal end of the lateral openings 52 and 54.

Embodiments that present various means for closing the écraseur loop 90 are presented below. The open loop écraseurs are referred to collectively and generically by reference character 30, and individually or specifically by a letter suffix (e.g., “open loop écraseur loop 30a”). This convention of using the same letter suffix to relate specific versions of an otherwise general component may be implemented for various components of the open loop écraseurs 30. For example, central stems are referred to collectively and generically by reference character 30, but specific aspects may be specified by the letter suffix corresponding to the associated open loop écraseur (e.g., “central stem 34b” corresponds to the central stem 34 of the open loop écraseur 30a). This convention is used herein, for example, for the central stems 34, snare assemblies 110, and internal bypasses 80 and their associated bypass axis 79.

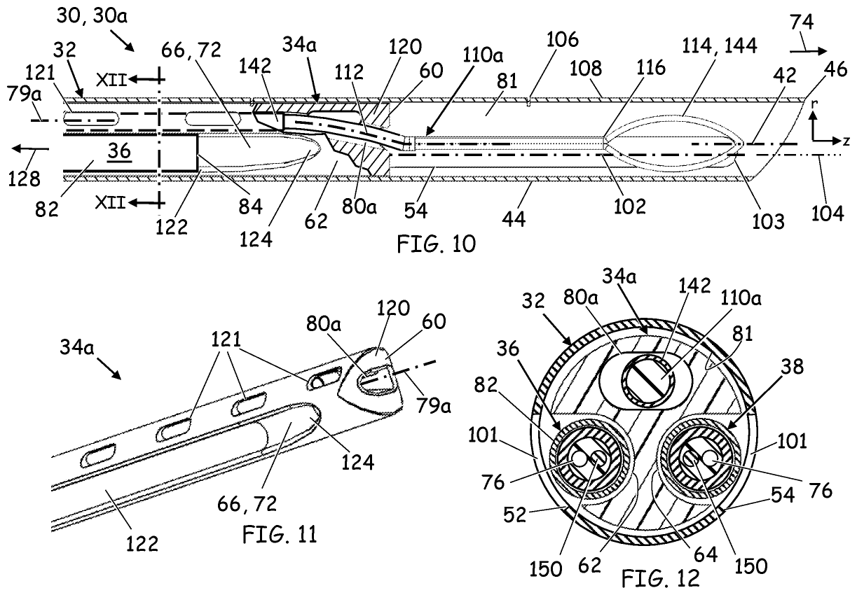

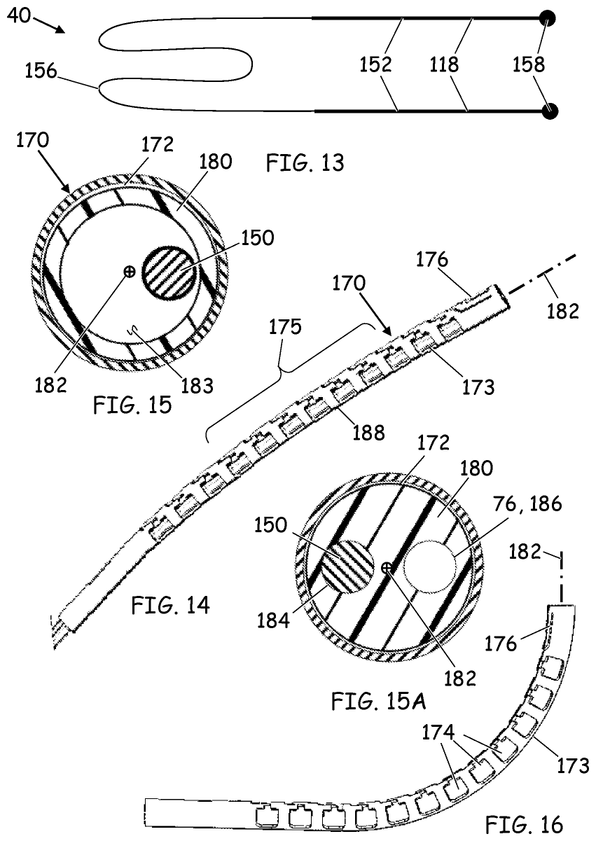

Referring to FIGS. 10 through 20, an open loop écraseur 30a is depicted according to embodiments of the disclosure. The open loop écraseur 30a includes the same components and attributes discussed attendant to FIGS. 2 through 5, which are identified by same-labeled reference characters. The open loop écraseur 30a includes at least one snare assembly 110a that defines a snare axis 112. Each of the at least one snare assembly 110a includes a snare portion 114 at a distal end 116 for coupling with an end portion 118 of the cutting wire assembly 40 (FIG. 13).

The open loop écraseur 30a may include an internal bypass 80a. The snare assembly 114 may be disposed and translatable within the internal bypass 80a. The internal bypass 80a is in fluid communication with the distal end portion 44 of the trocar 32 for translation of the snare assembly 110a into the distal end portion 44, between the lateral openings 52 and 54. In some embodiments, a bypass axis 79a of the internal bypass 80a is radially offset from the central axis 42 of the open loop écraseur 30a. The internal bypass 80a may include an end deflector 120 (depicted). In some embodiments, the internal bypass 80a may be tangentially offset relative to the lateral sides 62 and 64 of a central stem 34a (depicted). The central stem 34a may define a plurality of lateral through holes 121.

The lateral sides 62 and 64 may define side channels 122 that receive the steerable catheters 36 and 38. The side channels 122 cooperate with the interior surface 81 of the trocar 32 to enable free axial translation of the steerable catheters 36 and 38 therein. The side channels 122 may be separated (depicted) or defined by a lateral through-slot with the steerable catheters 36 and 38 in intermittent contact therein. The catheter deflector portions 66 and 68, when utilized, may be disposed at distal ends 124 of the side channels 122.

In some embodiments, the end portion 118 of the cutting wire assembly 40 is disposed in the working channel 76 of the steerable catheter 36. The end portion 118 may be extendible through the working channel 76 to exit the distal extremity 84 of the distal steering section 82 of the steerable catheter 36, 38 for coupling with the snare portion 114. The snare assembly 110a is retractable in a proximal direction 128 within the trocar 32 for at least partial circumscription of the cutting target 88.

In some embodiments, the snare assembly 110a includes a sleeve 142 that is selectively translatable over the snare axis 112 for collapsing and closure of the snare portion 114 about the end portion 118. Alternatively, translation of the central stem 34a over the snare portion 114 is contemplated for the closure. The snare portion 114 may be elastically collapsible. In some embodiments, the snare portion 114 includes a basket portion 144. Other configurations are contemplated for the snare portion 114, including hook portion and collapsible jaw portions.

In some embodiments, each of the steerable catheters 36 and 38 may include a steering wire 150 that is anchored to one side of the distal steering section 82 proximate the distal extremity 84. The distal steering section 82 may be rotationally oriented so that the anchored side is adjacent the respective lateral side 62, 64 of the central stem 34a. The steering wire 150 may extend along the interior surface 81 of the steerable catheter 36, 38 (FIG. 12). By this arrangement, applying a tension force to the steering wire 150 causes the distal extremity 84 of the steerable catheter 36, 38 to deflect radially inward, towards the central axis 42. In some embodiments, applying a compression force to the steering wire 150 causes the distal extremity 84 to deflect radially outward, away from the central axis 42, at least enough to start the path around the targeted organ or tissue 86.

In some embodiments, the end portion 118 of the cutting wire assembly 40 includes a guide wire portion 152 at a free end 154 that is attached to a cutting wire portion 156 (FIG. 13). The guide wire portion 152 may be more stout than the cutting wire portion 156, for example by increased cross-sectional diameter and/or more rigid material properties. In some embodiments, the guide and cutting wire portions 152 and 156 may be one unitary wire with different surface properties for the guide wire and cutting wire portions 156 and 158. The end portion 118 may include a head portion 158 that having a dimension that is greater than the diameter of guide and/or cutting wires 152, 156.

Functionally, the guide wire portion 152 facilitates advancement (pushing) of the end portions 118 through the working channels 76. The head portions 158 may facilitate capture of the end portions 118 by the snare portion 114, and may also serve to inhibit inadvertent retraction of the end portions 118 back into the working channels 76. In some embodiments, the end deflector 120 deflects the snare portion 114 of the snare assembly 110a towards the central axis 42, so that the snare portion 114 is substantially centered about the plane 104. The elongated geometry enables the lateral openings 52, 54 to serve as both egress and ingress for the steerable catheters 36, 38.

In some embodiments, the distal steering section 82 includes a slotted tube 170 (FIGS. 14 through 16). The slotted tube 170 defines a main lumen 172 and may define a pattern of slots 174 that extend laterally from a spine 173 to define an open side 175 of the slotted tube 170. The slots 174 are dimensioned to enable a range of deflections relative to a straight orientation while still providing torsional rigidity. The pattern of the slots 174 may be uniform or may be adapted in dimension to offer a tailored bending radius. The pattern defined by the slots 174 may be tailored to modify the flexibility along the length of the slotted tube 170 for a uniform articulate radius along the slotted tube 170. A non-limiting example of such a slotted tube 170 is found at U.S. Pat. No. 9,795,765 to Romoscanu, the disclosure of which is hereby incorporated by reference herein in its entirety except for patent claims and express definitions contained therein. The slotted tube 170 is adapted for lateral flexing by exerting a tension on the steering wire 150, the steering wire 150 being affixed proximate a distal end 176 of the slotted tube 170.

A flexible interior shaft 180 may be disposed within the main lumen 172 of the slotted tube 170. The flexible interior shaft 180 defines and is co-linear with a shaft axis 182 and may define a single lumen, double lumens, or more than two lumens. The steering wire 150 may be situated inside or outside the flexible interior shaft 180, depending on the chosen structure. Single lumen embodiments may define a substantially open cross-sectional area 183 (FIG. 15) for the working channel 76, through which the steering wire 150 is routed with the balance of the cross-sectional area 183 being available for passage of larger components, for example snare portions 114. Embodiments where the flexible interior shaft 180 defines two or more lumens (FIG. 15A) may include a steering wire lumen 184 and a working channel lumen 186. The steering wire lumen 184, within which the steering wire 150 is disposed, may be laterally offset from the shaft axis 182, with the flexible interior shaft 180 arranged within the slotted tube 170 so that the steering wire lumen 184 is disposed between the shaft axis 182 and the open side 175 of the slotted tube 170. The working channel lumen 186 defines the portion of the working channel 76 that extends through the flexible interior shaft 180.

Functionally, the slotted tube 170 provides primarily a one-sided lateral deformation, axial stiffness, and torsional stiffness. The slotted tube 170 also controls the distal steering section 82 of the steerable catheters 36, 38 through a planar articulation of known arc radius. Application of a tension force on the steering wire 150 causes the slotted tube 170 to flex laterally towards the open side 175 of the slotted tube 170. In some embodiments, a constant force is applied on the steering wire 150 to maintain the distal steering section 82 in a steered or arcuate configuration. The spine 173 may provide biasing of the slotted tube 170 toward a substantially straight configuration.

Super-elastic materials such as NITINOL® may be utilized. Iin some embodiments, thermal treatment of such super-elastic materials is implemented to configure the slotted tube 170 and distal steering section 82 to define an elastically bowed profile 188 that is substantially coplanar with the spine 173 and the shaft axis 182. The elastically bowed profile may also bias the slotted tube 170 (and subsequently the distal steering section 82) toward the spine 173. The distal steering section 82 may be oriented so that the elastically bowed profile 188 bends away from the central axis 42 of the trocar 32 to assist in the routing of the steerable catheters 36, 38 through the lateral openings 52 and 54.

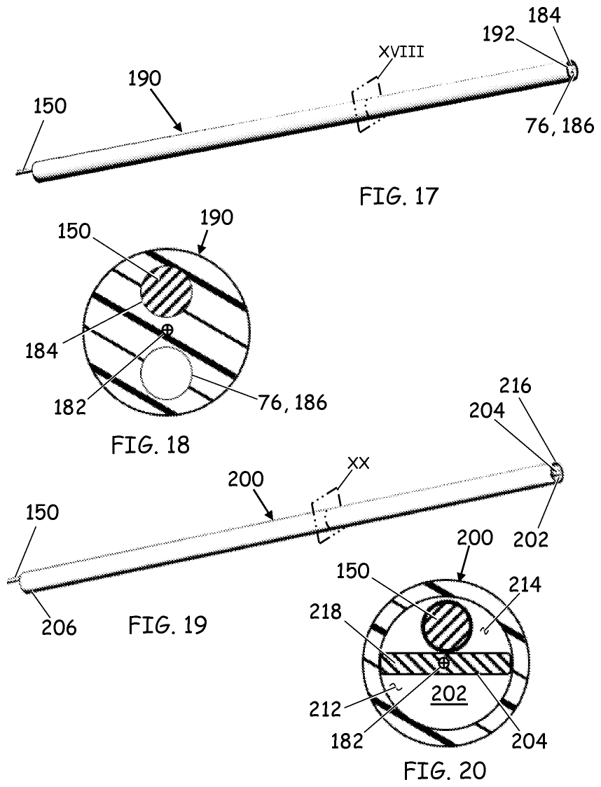

In some embodiments, the distal steering section 82 includes a multi-lumen flexible shaft 190 (FIGS. 17, 18) for use without the slotted tube 170. The multi-lumen flexible shaft 190 may include some of the same components and attributes as the flexible interior shaft 180, some of which are indicated by same-labeled reference characters. The steering wire 150 is coupled to a distal end 192 of the multi-lumen flexible shaft 190 and may be embedded within the matrix of the multi-lumen flexible shaft 190 for formation of the steering wire lumen 184.

Steering of the multi-lumen flexible shaft 190 may be accomplished by applying tension or compression to the steering wire 150. The deformation characteristics for steering are attained by the material properties of the multi-lumen flexible shaft 190. The embedded braiding provides axial stiffness for distal translation (pushing) of the steerable catheter 36, 38 within the trocar 32.

In some embodiments, the distal steering section 82 includes a single lumen shaft 200 that defines a central lumen 202 (FIGS. 19 and 20). A leaf spring 204 may be disposed in the central lumen 202, extending to a proximal end 206 of the single lumen shaft 200 to define separate passages 212 and 214 for the working channel 76 and the steering wire channel 78, respectively. The steering wire 150 is attached within or distally proximate the passage 214 near a distal end 216 of the single lumen shaft 200. The single lumen shaft 200 may be a polymer, such as polyether block amides, polyurethanes, and polyamides, for example formed by an extrusion process. The leaf spring 204 may define a rectangular cross-section 218 and may be fabricated from a metallic or composite material, for example stainless steel, ELGILOY®, PHYNOX®, FWM® 1058, NITINOL®, and carbon fiber reinforced polymers.

Functionally, application of a tension or compression force on steering wire 150 causes the single lumen shaft 200 to exert a bending moment along leaf spring 204 for a desired deflection or steering. The leaf spring 204 imposes anisotropic bending characteristics on the single lumen shaft 200, causing predictable planar deflection of the steerable catheter 36, 38.

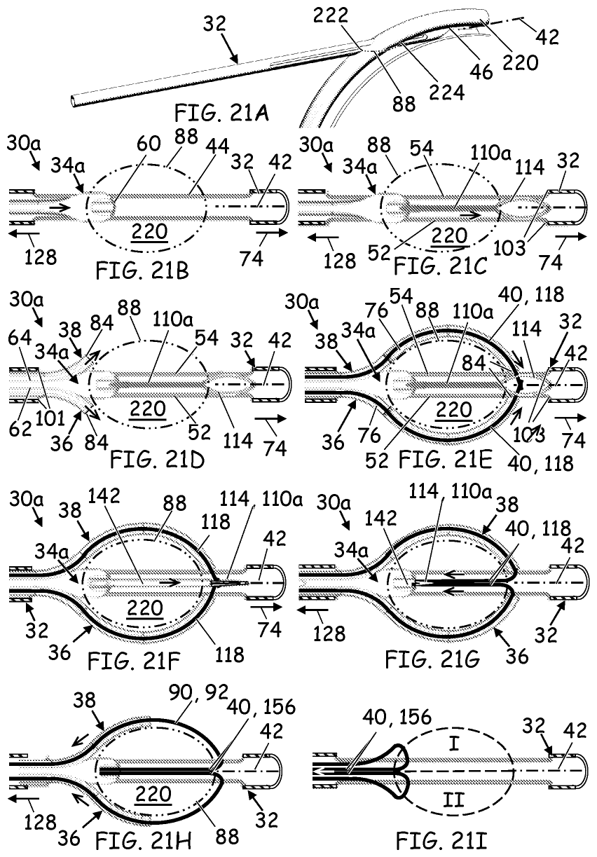

Referring to FIGS. 21A through 21I, operation of the open loop écraseur 30a in a tenotomy procedure is depicted according to an embodiment of the disclosure, wherein the targeted organ or tissue 86 is a tendon 220. The open loop écraseur 30a effects the bifurcated loop configuration 92 of FIGS. 3 and 5 (FIG. 21H). As such, two cutting zones I and II are effectuated (FIG. 21I).

The trocar 32 is introduced and passed through the tendon 220, entering at a puncture point 222 and exiting at an exit point 224 along the central axis 42 of the trocar (FIG. 21A). The puncture point 222 and exit point 224 define points on the perimeter of the cutting target 88. Insertion of the trocar 32 may be guided using conventional techniques such as ultrasound, X-ray, or visual (arthroscopic) control. The central stem 34a is inserted into the trocar 32 and translated along the central axis 42 so that the distal end 60 is within the distal end portion 44 of the trocar 32 and proximate the cutting target 88 (FIG. 21B). The snare assembly 110a is advanced through the internal bypass 80a so that the snare portion 114 is proximate the distal ends 103 of the lateral openings 52 and 54 (FIG. 21C). In some embodiments, the central stem 34a and snare assembly 110a are pre-positioned within the trocar 32 prior to puncturing of the tendon 220.

The steerable catheters 36, 38 are advanced in the distal direction 74 along the lateral sides 62, 64 of the central stem 34a. The distal extremities 84 of the steerable catheters 36, 38 are directed radially outward, away from the central axis 42, through the lateral openings 52, 54 near the proximal ends 101 (FIG. 21D). The initial exodus of the distal extremities 84 may be accomplished in one or a combination of several ways. In some embodiments, the distal extremities 84 are deflected radially outwards by the catheter deflector portions 66, 68 of the central stem 34a (depicted). In some embodiments, the elastically bowed profile 188 of the slotted tube 170 biases the distal extremities 84 of the steerable catheters 36, 38 against the interior surface 81 of the trocar 32, so that upon reaching the proximal ends 101 of the lateral openings 52, 54 the distal extremities 84 are biased radially outward and through the lateral openings 52, 54. In some embodiments, the steerable catheters 36, 38 can be steered radially outward upon reaching the proximal ends 101 of the lateral openings 52, 54, for example by applying a compressive force to the steering wires 150.

Upon exiting the lateral openings 52, 54, advancement of the steerable catheters 36, 38 continues in the distal direction 74 with the distal extremities 84 being steered around the tendon 220 to approach or reenter the lateral openings 52, 54 proximate the distal ends 103. The end portions 118 of one or more cutting wire assemblies 40 are advanced through the working channels 76 so as to reenter the distal end portion 44 of the trocar 32 via the lateral openings 52, 54 and extend into or through the snare portions 114 (FIG. 21E).

Advancement and steering of the steerable catheters 36, 38 may be guided using conventional techniques such as ultrasound, X-ray, or visual (arthroscopic) control. In some embodiments, the end portions 118 are advanced or otherwise disposed proximate the distal extremities 84 prior to the distal advancement of the steerable catheters 36, 38. In some embodiments, the head portions 158 of the distal end portions 118 may be oversized to inhibit retraction into the working channels 76. The end portions 118 may be opposite ends of the same cutting wire assembly 40 or single end portions 118 of two separate cutting wire assemblies 40.

With the end portions 118 disposed in or through the snare portion 114, the sleeve 142 is advanced in the distal direction 74 at least partially over the snare portion 114, causing the snare portion 114 to collapse over and capture the end portions 118 (FIG. 21F). Having coupled the snare portion 114 to the end portions 118, the snare assembly 110a is retracted into the central stem 34a to draw the cutting wire assembly 40 through the trocar 32 in the proximal direction 128 (FIG. 21G). The steerable catheters 36, 38 are retracted (FIG. 21H) so that the distal extremities 84 are drawn into the trocar 32 proximal to the proximal ends 101 of the lateral openings 52, 54. For cutting wire assemblies 40 that include the guide wire portion 152, the cutting wire assembly 40 is drawn through the trocar 32 until both zones I and II of the cutting target 88 are circumscribed by the cutting wire portion 156. The central stem 34a may be retracted proximally, and the cutting wire portion 156 drawn through the zones I and II for severance of the tendon 220 (FIG. 21I). The cutting wire assembly 40 and open loop écraseur 30a are withdrawn from the cutting target 88 along the same path the trocar 32 was introduced.

The above-mentioned aspects are described in relation to the open loop écraseur 30a. In view of this disclosure, the skilled artisan will understand how to apply these and other aspects mutatis mutandis to other embodiments of the open loop écraseur 30 generally.

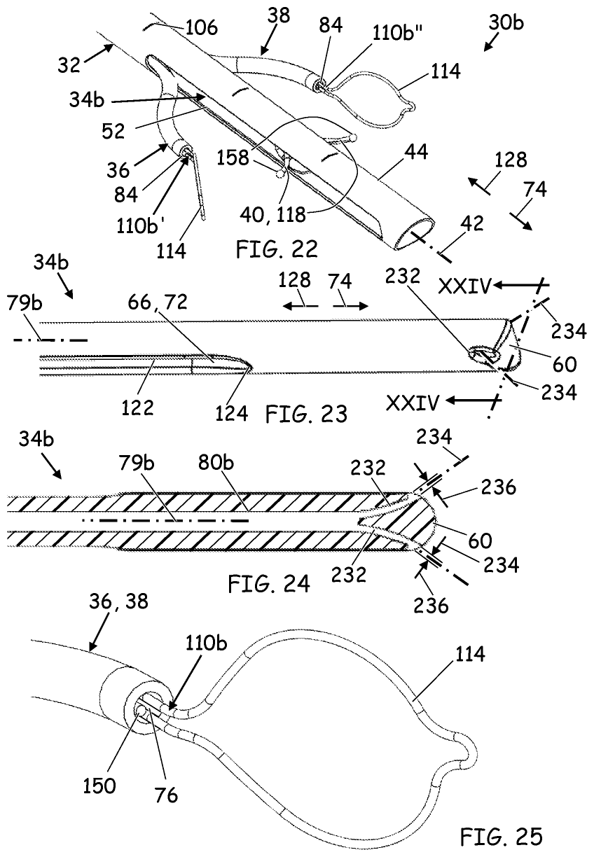

Referring to FIGS. 22 through 25, an open loop écraseur 30b is depicted according to embodiments of the disclosure. The open loop écraseur 30b includes many of the same components and attributes as the open loop écraseur 30a, which are identified by same-labeled reference characters. A distinction of the open loop écraseur 30b is that the cutting wire assembly 40 is disposed in an internal bypass 80b of the trocar 32 and the snare portions 114 are disposed in the steerable catheters 36 and 38. Accordingly, the open loop écraseur 30b includes a central stem 34b configured to receive and deploy the cutting wire assembly 40 and the steerable catheters 36 and 38 are configured to accommodate translation of the first and second snare assemblies 110b′ and 110b″ into and out of the distal extremities 84. The first and second snare assemblies 110b′ and 110b″ are referred to generically and collectively as snare assembly or assemblies 110b. Structural and operational aspects for closing the open loop écraseur 30b are described below, along with distinctions of the central stem 34b and snare assemblies 110b relative to the central stem 34a and the snare assembly 110a.

The snare assemblies 110b are translatable within the working channels 76 of the steerable catheters 36 and 38, respectively. Each snare assembly 110b′ and 110b″ includes the snare portions 114 at the distal ends 116 thereof for coupling with the end portions 118 of one or more cutting wire assembly or assemblies 40. The snare portions 114 are extendible through the respective working channels 76 of the steerable catheters 36, 38 to exit the distal extremities 127 of the distal steering sections 82 of the steerable catheters 36 and 38. The resilient nature of the snare portions 114 cause the snare portions 114 to open upon exiting the distal extremity 84 of the steerable catheters 36, 38. The snare portions 114 can also be retracted proximally through the distal extremities 84 of the steerable catheters 36, 38. Reentry into steerable catheters 36, 38 causes the snare portions to collapse over and capture the end portions 118 of the cutting wire assembly 40. In some embodiments, the steering wires 150 occupy the working channels 76 without dedicated lumens or passages (FIG. 25), at least proximate the distal extremities 84, thus providing ample cross-section for distal and proximal translation of the snare portions 114 through the distal extremities 84.

In some embodiments, the central stem 34b defines exit ports 232 proximate the distal end 60 thereof, the exit ports 232 being in fluid communication with the internal bypass 80b defined within the trocar 32. The internal bypass 80b extends axially and proximally from the exit ports 232, the exit ports 232 being in fluid communication with the distal end portion 44 of the trocar 32. Each exit port 232 defines an exit port axis 234 that extends radially outward in the distal direction 74 for deflection of the end portions 118 through the lateral openings 52 and 54 of the trocar 32 for coupling with the respective snare portions 114.

The end portions 118 of the cutting wire assembly or assemblies 40 are disposed within the internal bypass 80b and extend through the respective exit ports 232. The snare assemblies 110b and the central stem 34b are retractable in the proximal direction 128 to at least partial circumscribe the cutting target 88. In some embodiments, the end portions 118 of the cutting wire assembly or assemblies 40 include the head portions 158 that are larger than an inner diameter 236 of the exit port 232.

Functionally, the arrangement of the snare assemblies 110b and the cutting wire assembly 40 enables the snare portions 114 to be positioned adjacent and exterior to the respective lateral openings 52 and 54 of the trocar 32 in an open configuration that can receive the end portions 118 of the cutting wire assembly 40. During retraction back into the steerable catheters 36, 38, the interaction between the snare portions 114 of the snare assemblies 110b and the steerable catheters 36, 38 is akin to the interaction between the snare portion 114 of the snare assembly 110a and the sleeve 142 of the open loop écraseur 30a.

The canted configuration of the exit port axes 234 relative to the central axis 42 acts to deflect the end portions 118 of the cutting wire assembly 40 radially outward, through the lateral openings 52, 54. For embodiments including portions 158 that are larger than the inner diameters 236 of the exit ports 232, inadvertent retraction of the end portions 118 back through the exit ports 236 may be inhibited.

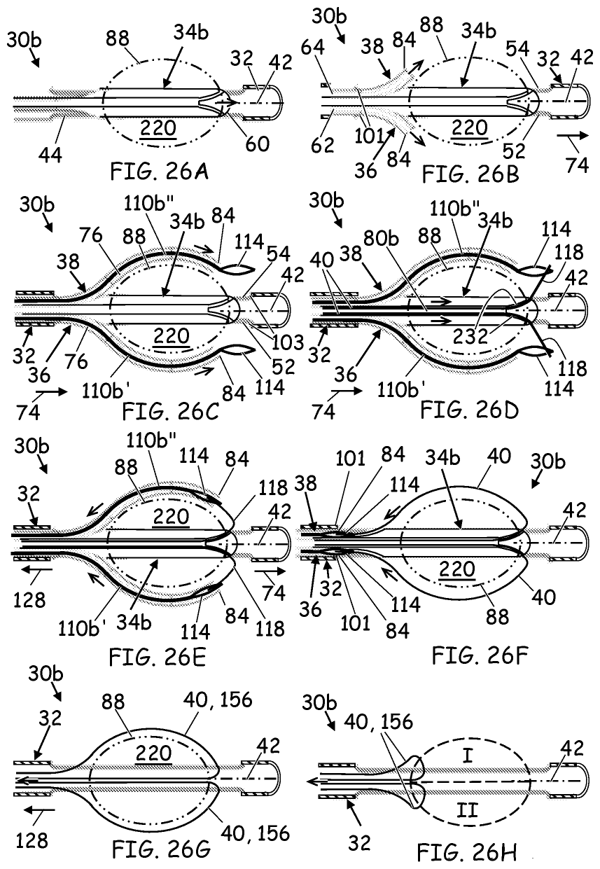

Referring to FIGS. 26A through 26H, operation of the open loop écraseur 30b in a tenotomy procedure is depicted according to an embodiment of the disclosure. As with the operation of open loop écraseur 30a (FIGS. 21A through 21I), the targeted organ or tissue 86 is the tendon 220. The open loop écraseur 30b also effects the bifurcated loop configuration 92 of FIGS. 3 and 5. As such, two cutting zones I and II are effectuated (FIG. 26H).

As in FIG. 21A, the trocar 32 may be introduced and passed through the tendon 220, entering at the puncture point 222 and exiting at the exit point 224 along the central axis 42 of the trocar 32. The central stem 34b is inserted into the trocar 32 and translated along the central axis 42 so that the distal end 60 is within the distal end portion 44 of the trocar 32 and into or through the cutting target 88 (FIG. 26A). Alternatively, the central stem 34b may be pre-positioned within the trocar 32 prior to puncturing of the tendon 220.

The steerable catheters 36, 38 are advanced in the distal direction 74 along the lateral sides 62, 64 of the central stem 34b. The distal extremities 84 of the steerable catheters 36, 38 are directed radially outward, away from the central axis 42, through the lateral openings 52, 54 near the proximal ends 101 (FIG. 26B). The initial exodus of the distal extremities 84 may be accomplished by deflection of the steerable catheters 36, 38 with the deflector portions 66, 68 (depicted) or in one or a combination the of the several ways described attendant to FIG. 21D.

Upon exiting the lateral openings 52, 54, advancement of the steerable catheters 36, 38 continues in the distal direction 74 with the distal extremities 84 being steered around the tendon 220 to approach the lateral openings 52, 54 proximate the distal ends 103. The snare assemblies 110b are advanced through the working channels 76 so that the snare portions 114 to exit the distal end portions 84 of the steerable catheters 36, 38 to open the snare portions 114. The open snare portions 114 are positioned exterior to and adjacent the lateral openings 52, 54 (FIG. 26C).

The cutting wire assembly or assemblies 40 are advanced through the exit ports 236 so that the end portions 118 pass through the open snare portions 114 (FIG. 26D). In some embodiments, the end portions 118 are also translated through the internal bypass 80b during this step. Alternatively, the distal ends 118 may be disposed within the exit ports 236 prior to inserting the central stem 34b into the trocar 32.

With the end portions 118 extended through the snare portion 114, the snare portions 114 are collapsed within the steerable catheters 36, 38 (FIG. 26E). The collapsing step may be accomplished by translating the snare assemblies 110b in the proximal direction 128 through the distal extremities 84 (depicted). Alternatively, the steerable catheters 36, 38 may be advanced in the distal direction 74 with the snare assemblies 110b held stationary, so that the distal extremities 84 advance over the snare portions 114 to collapse the snare portions 114.

Having coupled the snare portions 114 to the end portions 118 within the distal steering sections 82, the steerable catheters 36, 38 are retracted into the trocar 32 so that the distal extremities 84 are drawn into the trocar 32 proximal to the proximal ends 101 of the lateral openings 52, 54 (FIG. 26F). For cutting wire assemblies 40 that include the guide wire portion 152, the cutting wire assembly 40 is drawn through the trocar 32 until both zones I and II of the cutting target 88 are circumscribed by the cutting wire portion 156. The central stem 34b may be retracted in the proximal direction 128 (FIG. 26G), and the cutting wire portion 156 drawn through the zones I and II for severance of the tendon 220 (FIG. 26G). The cutting wire assembly 40 and open loop écraseur 30a are withdrawn from the cutting target 88 along the same path the trocar 32 was introduced.

The open loop écraseurs 30a and 30b may be utilized to reify the single asymmetric loop configuration 96 of FIGS. 6 and 8. The skilled artisan, in view of this disclosure, understands that by closing the loop with only the distal end portion 118 of the cutting wire assembly 40, the asymmetric loop configuration 96 and partial severance of the targeted tendon 220 or organ 86 can be effectuated. In some embodiments, the open loop écraseurs 30a and/or 30b are structured only to effectuate the single loop configuration 96. That is, the trocar 32 and central stem 34a, 34b may be configured to receive and route only the first steerable catheter 36, for example by inclusion of only deflector portion 66 and the presence of only lateral opening 52. Such an arrangement is depicted at FIGS. 8 and 9, and is readily implemented, in view of this disclosure, by the skilled artisan.

The asymmetric loop configuration 96 may also be utilized to effect a complete severance of the targeted tendon 220 or organ 86. That is, by introducing the trocar 32 either adjacent the tendon/organ 220, 86 or with an off center (grazing) puncture, the cutting target 88 of the asymmetric loop configuration 96 can provide either total severance or severance of the remnant cross-section from a grazing introduction.

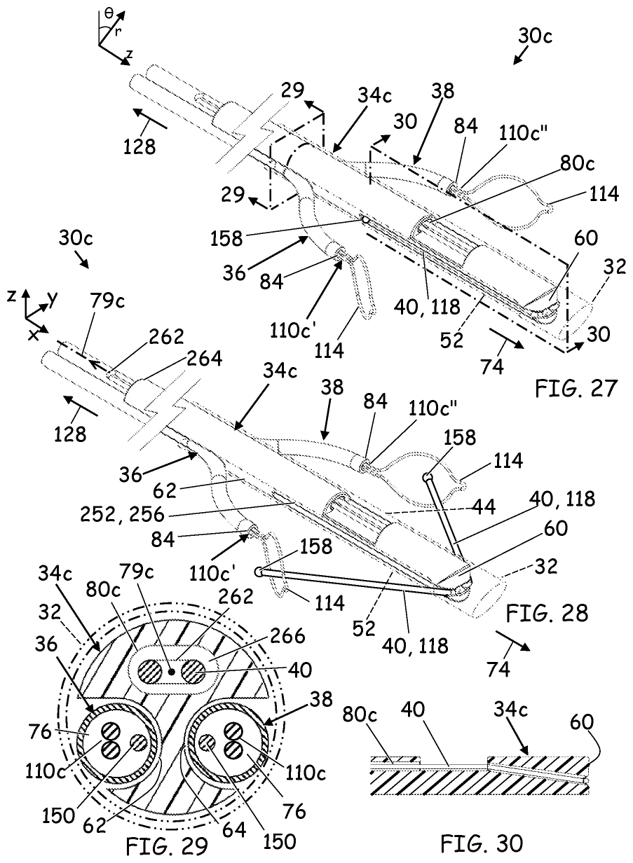

Referring to FIGS. 27 through 30, an open loop écraseur 30c is depicted according to an embodiment of the disclosure. The open loop écraseur 30c includes many of the same components and attributes as the open loop écraseur 30b, which are identified by same-labeled reference characters. A distinction of the open loop écraseur 30c is that a central stem 34c defines an internal bypass 80c within the trocar 32 (depicted in phantom in FIGS. 27 and 28) to extend axially for fluid communication with the distal end portion 44 of the trocar 32. Structural and operational aspects for closing the open loop écraseur 30c are described below, along with distinctions of the central stem 34c relative to the central stems 34a, 34b. For the depicted embodiment, the snare assemblies 110c are the same as the snare assemblies 110b.

The end portions 118 of the cutting wire assembly 40 are disposed within the internal bypass 80c, extend into the distal end portion 44 of the trocar 32, and around the distal end 60 of the central stem 34c. The end portions 118 extend proximal to the distal end 60 along the lateral sides 62, 64 of the central stem 34c and adjacent the lateral openings 52, 54 of the trocar 32 (FIG. 27). When the cutting wire assembly 40 is translated along a bypass axis 79c in the proximal direction 128, the end portions 118 are pulled around the distal end 60 of the central stem 34c and, because of the stiffness of the wire, deflect radially outward, through the lateral openings 52, 54 (FIG. 28).

The snare portions 114 of the snare assemblies 110c are positioned the receive the deflected end portions 118 for circumscription of the cutting target 88. The snare assemblies 110c may be of the same construction and functionality as the snare assemblies 110b of the open loop écraseur 30b.

The end portions 118 of the cutting wire assembly 40 may be disposed in axial channels 252 defined along exterior surfaces 254 of the lateral sides 62, 64 of the central stem 34c. In some embodiments, the end portions 118 of the cutting wire assembly 40 are frangibly attached to the lateral sides 62, 64 of the central stem 34c. Herein, a “frangible” attachment is one that is easily broken, for example by a low strength glue or a light interference fit. In some embodiments, the end portions 118 include the head portions 158 sized for an interference fit within notches 256 (e.g., the axial channels 252) formed on respective sides 62, 64 of the central stem 34c. When the cutting wire assembly 40 is pulled around the distal end 60 of the central stem, a bending moment is imparted on the segments of the end portions 118 that extend along the exterior surfaces 254. The bending moment overcomes the frangible attachment, enabling the end portions 118 to deflect radially outward.

In the depicted embodiment, the open loop écraseur 30c operates to provide the single symmetrical loop configuration 94. Specifically, the open loop écraseur 30c is configured so that the end portions 118 are two ends of the same cutting wire assembly 40. The cutting wire assembly 40 is substantially centered about a bend 262 that may extend proximally from a proximal end 264 of the central stem 34c. The two end portions 118 extend distal to the bend 262 for routing through the internal bypass 80c, around the distal end 60 of the central stem 34c, and along the respective lateral sides 62, 64. Unlike the internal bypass 80b, the internal bypass 80c does not branch into separate channels proximal to the distal end 60 of the central stem 34c. Rather, the internal bypass 80c defines a single passage 266 (FIGS. 28 and 29) that extends through the distal end 60 of the central stem 34c. As such, when the steerable catheters 36 and 38 are retracted in the proximal direction 128 after coupling with the end portions 118, the bend 262 advances distally through the single passage 266 and exits the distal end 60 of the central stem 34c. In this way, the cutting wire assembly 40 forms the écraseur loop 90 of the single symmetrical loop configuration 94.

The open loop écraseur 30c can also be implemented to effect the closed loop configurations 94 or 96. For example, proximal retraction of the end portions 118 can be ceased before the bend 262 exits the distal end 60 of the central stem 34, followed by proximal retraction of the bend 262 along with the end portions 118. Also, the end portions may be from two separate cutting wire assemblies 40 to operate akin to the open loop écraseur 30b. Such operation would create the bifurcated loop configuration 92 of FIGS. 3 and 5. In another example, only one end portion 118 is advanced through the stem 34c, again akin to the open loop écraseur 30b for the asymmetric loop configuration 96.

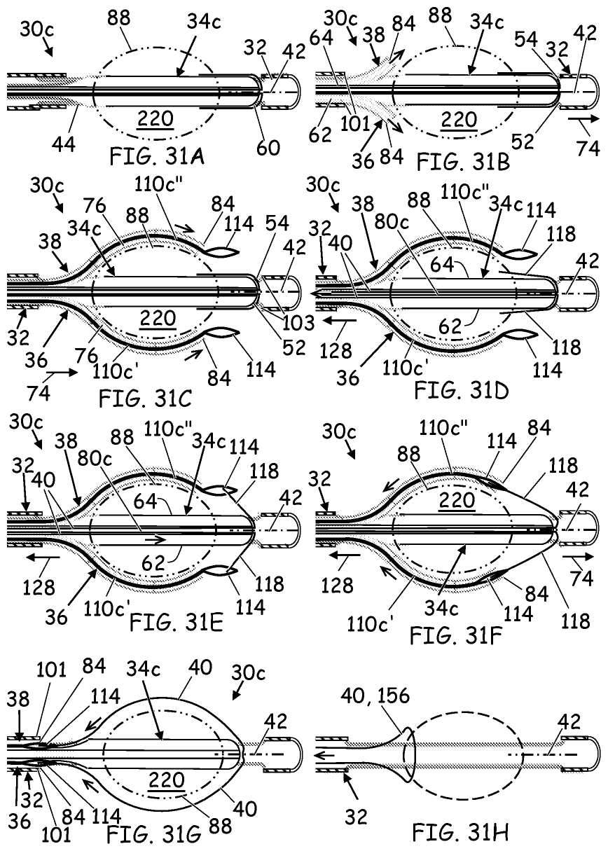

Referring to FIGS. 31A through 31H, operation of the open loop écraseur 30c in a tenotomy procedure is depicted according to an embodiment of the disclosure. As with the operation of open loop écraseur 30b (FIGS. 26A through 26H), the targeted organ or tissue 86 is the tendon 220. The open loop écraseur 30c effects the single symmetrical loop configuration 94 of 5 and 6.

The central stem 34c and cutting wire assembly 40 is first prepared as depicted in FIG. 27. As in FIG. 21A, the trocar 32 may be introduced and passed through the tendon 220, entering at the puncture point 222 and exiting at the exit point 224 along the central axis 42 of the trocar 32. The central stem 34c is inserted into the trocar 32 and translated along the central axis 42 so that the distal end 60 is within the distal end portion 44 of the trocar 32 and passes through the cutting target 88 (FIG. 31A). Alternatively, the central stem 34c may be pre-positioned within the trocar 32 prior to puncturing of the tendon 220.

The steerable catheters 36, 38 are advanced in the distal direction 74 along the lateral sides 62, 64 of the central stem 34b. The distal extremities 84 of the steerable catheters 36, 38 are directed radially outward, away from the central axis 42, through the lateral openings 52, 54 near the proximal ends 101 (FIG. 31B). The initial exodus of the distal extremities 84 may be accomplished by deflection of the steerable catheters 36, 38 with the deflector portions 66, 68 (depicted) or in one or a combination the of the several ways described attendant to FIG. 21D.

Upon exiting the lateral openings 52, 54, advancement of the steerable catheters 36, 38 continues in the distal direction 74 with the distal extremities 84 being steered around the tendon 220 to approach the lateral openings 52, 54 proximate the distal ends 103. The snare assemblies 110c are advanced through the working channels 76 so that the snare portions 114 to exit the distal end portions 84 of the steerable catheters 36, 38 to open the snare portions 114. The open snare portions 114 are positioned exterior to and adjacent the lateral openings 52, 54 (FIG. 31C).

The cutting wire assembly or assemblies 40 are retracted in the proximal direction 128, causing the end portions 118 to break free from the frangible connection of the lateral sides 62, 64 (FIG. 31D). Initially, the end portions 118 may still be captured within the tendon 220, preventing the end portions 118 from entering the snare portions 114 of the snare assemblies 110c. The retraction of the cutting wire assembly 40 in the proximal direction may continue until the end portions 118 are clear of the tendon 220, enabling the end portions 118 to deflect radially outward and into the snare portions 114 (FIG. 31E). Alternatively, the stem portion 34 may be translated distally to clear the tendon 220, then retracted proximally so that the end portions 118 enter the snare portions 114.

With the end portions 118 extended through the snare portion 114, the snare portions 114 are collapsed within the steerable catheters 36, 38 (FIG. 31F). The collapsing step may be accomplished by translating the snare assemblies 110c in the proximal direction 128 through the distal extremities 84 (depicted). Alternatively, the steerable catheters 36, 38 may be advanced in the distal direction 74 with the snare assemblies 110c held stationary, so that the distal extremities 84 advance over the snare portions 114 to collapse the snare portions 114.

Having coupled the snare portions 114 to the end portions 118 within the distal steering sections 82, the steerable catheters 36, 38 are retracted into the trocar 32 so that the distal extremities 84 are drawn into the trocar 32 proximally beyond the proximal ends 101 of the lateral openings 52, 54 (FIG. 31G). This action also causes the bend 262 to exit the distal end 60 of the central stem 34. The central stem 34c is retracted in the proximal direction 128 and the cutting wire portion 156 drawn through the cutting target 88 for severance of the tendon 220 (FIG. 31H). The cutting wire assembly 40 and open loop écraseur 30a are withdrawn from the cutting target 88 along the same path the trocar 32 was introduced.

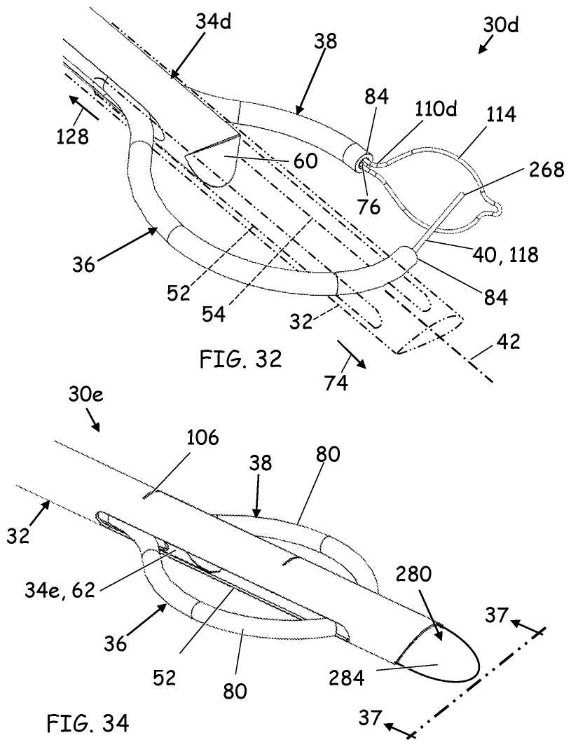

Referring to FIG. 32, an open loop écraseur 30d is depicted according to an embodiment of the disclosure. The open loop écraseur 30c includes many of the same components and attributes as the open loop écraseurs 30a and 30c, which are identified by same-labeled reference characters. A distinction of the open loop écraseur 30d is that both a snare assembly 110d and the cutting wire assembly 40 are routed through the steerable catheters 36, 38. As such, a central stem 34d doesn't require provisions for the internal bypass 80. Structural and operational aspects for closing the open loop écraseur 30d are described below. The central stem 34d may be structured the same as the central stems 30a and 30b. In one embodiment, the internal bypasses 80a, 80b are eliminated (depicted). For the depicted embodiment, the snare assembly 110d is the same as the snare assemblies 110b.

A distal extremity 268 of the end portion 118 of the cutting wire assembly 40 is routed through both the lateral openings 52 and 54 for coupling with the snare portion 114 of the snare assembly 110d. The snare portion 114 is coupled to the end portion 118, and the snare assembly 110d is retracted in the proximal direction 128 to draw the cutting wire assembly 40 into the working channel 76 of the steerable catheter 38 for at least partial circumscription of the cutting target 88. The distal extremity 268 may include the head portion 158, such as depicted at FIG. 13.

In some embodiments the distal steering section 82 of the steerable catheter 36 is steerable through the central axis 42 of the trocar 32 for routing of the distal extremity 268 of the end portion 118 of the cutting wire assembly 40 through the lateral openings 52 and 54, the steerable catheter 36 and the lateral openings 52 and 54 being dimensioned to enable such routing. In some embodiments, the end portion 118 of the cutting wire assembly 40 is extendible from the distal extremity 84 of the steerable catheter 36 for routing the end portion 118 through the lateral openings 52 and 54. By this arrangement, the lateral openings 52 and 54 do not need to be dimensioned to receive the steerable catheter 36.

As with the open loop écraseurs 30b and 30c, collapsing the snare portion 114 for coupling to the end portion 118 may be accomplished by translating the snare assembly 110d proximally through the distal extremities 84 or by advancing the steerable catheter 38 distally with the snare assembly 110d held stationary, thereby advancing the distal extremity 84 of the steerable catheter 38 over the snare portion 114.

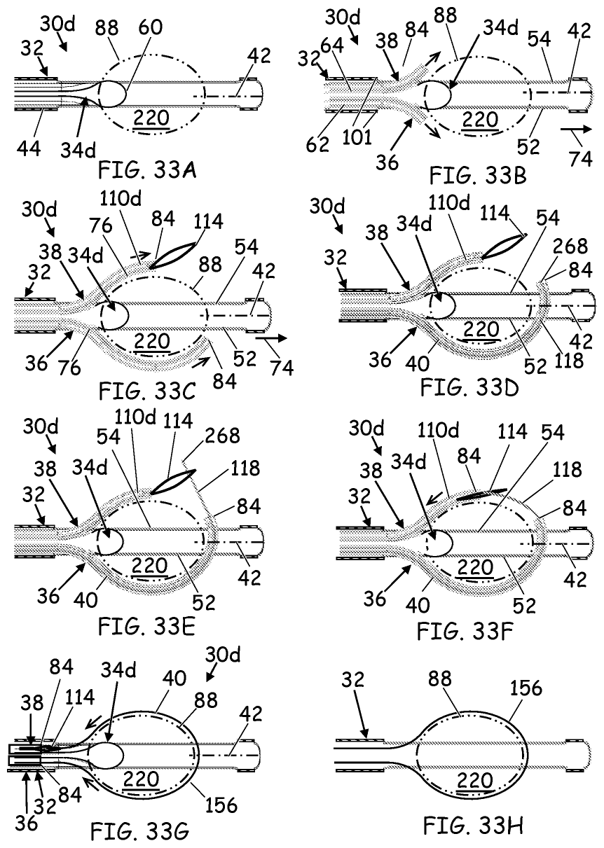

Referring to FIGS. 33A through 33H, operation of the open loop écraseur 30d in a tenotomy procedure is depicted according to an embodiment of the disclosure. As with the operation of open loop écraseur 30a (FIGS. 21A through 21I), the targeted organ or tissue 86 is the tendon 220. The open loop écraseur 30d effects the single symmetrical bifurcated loop configuration 94 of FIGS. 6 and 7.

As in FIG. 21A, the trocar 32 may be introduced and passed through the tendon 220, entering at the puncture point 222 and exiting at the exit point 224 along the central axis 42 of the trocar 32. The central stem 34d is inserted into the trocar 32 and translated along the central axis 42 so that the distal end 60 is within the distal end portion 44 of the trocar 32 and proximate the cutting target 88 (FIG. 33A). Alternatively, the central stem 34d may be pre-positioned within the trocar 32 prior to puncturing of the tendon 220.

The steerable catheters 36, 38 are advanced in the distal direction 74 along the lateral sides 62, 64 of the central stem 34d. The distal extremities 84 of the steerable catheters 36, 38 are directed radially outward, away from the central axis 42, through the lateral openings 52, 54 near the proximal ends 101 (FIG. 33B). The initial exodus of the distal extremities 84 may be accomplished by deflection of the steerable catheters 36, 38 with the deflector portions 66, 68 (depicted) or in one or a combination the of the several ways described attendant to FIG. 21D.

Upon exiting the lateral openings 52, 54, advancement of the steerable catheters 36, 38 continues in the distal direction 74 with the distal extremities 84 being steered around the tendon 220 to approach the lateral openings 52, 54 proximate the distal ends 103. The snare assembly 110d is advanced through the working channel 76 of the steerable catheter 38 so that the snare portion 114 to exits the distal end portion 84 of the steerable catheter 38 to open the snare portions 114. The snare assembly 110d is positioned so that the open snare portion 114 is exterior to and adjacent the lateral opening 54 (FIG. 33C).

The distal end 118 of the cutting wire assembly 40 is advanced through the lateral openings 52 and 54 (FIG. 33D). In the depicted embodiment, this step is accomplished by positioning the distal extremity 268 of the of the end portion 118 of the cutting wire assembly 40 proximate the distal extremity 84 of the steerable catheter 36 and steering the distal extremity 84 through the lateral openings 52 and 54. In an alternative and undepicted arrangement, the distal extremity 84 of the steerable catheter 36 may be positioned outside the trocar 32 or inserted through only the lateral opening 52 and the end portion 118 extended beyond the distal extremity 84 to pass through both lateral openings 52 and 54.

The end portion 118 is further advanced through the steerable catheter 36 and distal extremity 84 to enter the open snare portion 114 (FIG. 33E). With the end portion 118 extended through the snare portion 114, the snare portions 114 is collapsed within the steerable catheter 38 (FIG. 33F). The collapsing step may be accomplished by translating the snare assemblies 110d in the proximal direction 128 through the distal extremity 84 of the steerable catheter 38 (depicted). Alternatively, the steerable catheter 38 may be advanced in the distal direction 74 with the snare assembly 110d held stationary, so that the distal extremity 84 advances over the snare portion 114 to collapse the snare portions 114.

Having coupled the snare portion 114 to the end portion 118 within the distal steering section 82 of the steerable catheter 38, the steerable catheters 36 and 38 are retracted into the trocar 32 so that the distal extremities 84 are drawn into the trocar 32 proximally beyond lateral openings 52, 54 (FIG. 33G), thereby fully exposing the cutting target 88 to the cutting wire assembly 40. For cutting wire assemblies 40 that include the guide wire portion 152, the steerable catheter 38 is drawn through the trocar 32 until the cutting target 88 is circumscribed by the cutting wire portion 156. The central stem 34d may be retracted in the proximal direction 128 (FIG. 33H), and the cutting wire portion 156 drawn through the cutting target for severance of the tendon 220 (see FIG. 31H). The cutting wire assembly 40 and open loop écraseur 30d are withdrawn from the cutting target 88 along the same path the trocar 32 was introduced.

The open loop écraseurs 30a and 30b may be utilized to reify the single asymmetric loop configuration 96 of FIGS. 6 and 7. The skilled artisan, in view of this disclosure, understands that by closing the loop with only the distal end portion 118 of the cutting wire assembly 40, the asymmetric loop configuration 96 and partial severance of the targeted tendon 220 or organ 86 can be effectuated. In some embodiments, the open loop écraseurs 30a and/or 30b are structured only to effectuate the single loop configuration 96. That is, the trocar 32 and central stem 34a, 34b may be configured to receive and route only the first steerable catheter 36, for example by inclusion of only deflector portion 66 and the presence of only lateral opening 52. Such an arrangement is depicted at FIGS. 8 and 9, and is readily implemented, in view of this disclosure, by the skilled artisan.

Operation of the open loop écraseurs 30a, 30b, 30c, and 30d are similar and based on the same principles as the operation of the open loop écraseur 30a. Specifically, all of the open loop écraseurs 30a-30d complete the coupling between cutting wire assemblies 40 and snare assemblies 110 to close the écraseur loops 90.

Referring to FIGS. 34 through 37, an open loop écraseur 30e is depicted according to an embodiment of the disclosure. The open loop écraseur 30e includes many of the same components and attributes as the other open loop écraseurs 30 disclosed herein, which are identified by same-labeled reference characters. A distinction of the open loop écraseur 30e is that closing the écraseur loop 90 does not involve snare assemblies. Rather, the cutting wire assembly 40 is routed directly from one steerable catheter 36 to the other steerable catheter 38. As such, a central stem 34e doesn't require accommodation of an internal bypass structure. Structural and operational aspects for closing the open loop écraseur 30e are described below.

The steerable catheters 36 and 38 are disposed in the trocar 32 along the lateral sides 62 and 64, respectively, of the central stem 34e. The end portion 118 of the cutting wire assembly 40 is disposed in the working channel 76 of the steerable catheter 36. The distal steering section 82 of the steerable catheter 36 is steerable through the lateral opening 52 and the distal steering section 82 of the steerable catheter 38 is steerable through the lateral opening 54 for lateral alignment of the distal extremities 84 of the steerable catheters 36 and 38 within the distal end portion 44 of the trocar 32. The end portion 118 of the cutting wire assembly 40 is extendible from the working channel 76 of the steerable catheter 36 into the working channel 76 of the steerable catheter 38 to at least partially circumscribe the cutting target 88 with the cutting wire assembly 40. The cutting wire is translated into the steerable catheter 38 from the steerable catheter 36 to complete the écraseur loop 90.

In some embodiments, a guide 280 is disposed within and dimensioned for sliding axial translation within the trocar 32. The guide 280 includes a hollow tubular portion 282 that extends to a distal alignment head 284, the hollow tubular 282 portion being dimensioned to receive the central stem 34e for sliding axial translation within the hollow tubular portion 282. The guide 280 defines first and second lateral openings 286 and 288 configured for tangential alignment with the first and second lateral openings 52 and 54, respectively, of the trocar 32.

The distal alignment head 284 includes a proximal face 292 that cooperates with a distal face 294 of the central stem 34e for selective clamping of the distal extremities 84 of the steerable catheters 36 and 38 within the distal end portion 44 of the trocar 32. A clamping configuration 296 is depicted at FIG. 37. In some embodiments, one or both of the proximal face 292 of the distal alignment head 284 and the distal face 294 of the central stem 34e define concave channels 298 (depicted for the distal face 294 of the central stem 34e at FIG. 36).

Functionally, the clamping configuration 296 laterally aligns the distal extremities 84 of the steerable catheters 36 and 38. The concave channels 296, when included, register against steerable catheters 36 and 38 in the clamping configuration 296 for vertical alignment of the distal extremities 84. The concave channel(s) 296 may define arcuate profiles (depicted) or otherwise define a concave geometry (e.g., polygonal profiles such as V-grooves or rectangular grooves).

In some embodiments, the cross sections of the steerable catheters 36 and 38 may be different. For example, steerable catheter 36, which conveys the cutting wire assembly 40 to steerable catheter 38, may include the working channel lumen 186 of FIGS. 15A and 18, whereas the steerable catheter 38 may define the substantially open cross-sectional area 183 of FIG. 15. In this way, the steerable catheter 36 accurately controls the trajectory of the cutting wire assembly 40 during entry into the steerable catheter 38 while the substantially open cross-sectional area 183 of the steerable catheter 38 provides a greater margin for receiving the end portion 118.

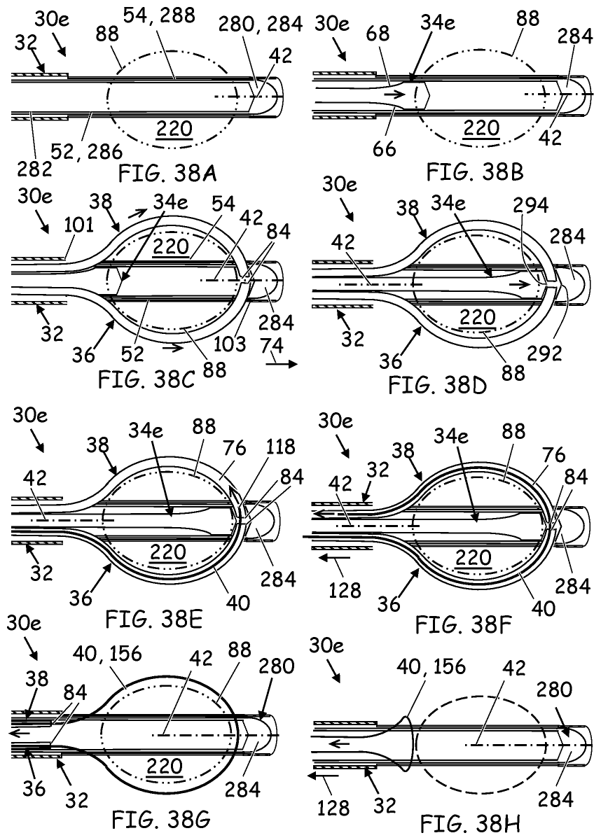

Referring to FIGS. 38A through 38H, operation of the open loop écraseur 30e in a tenotomy procedure is depicted according to an embodiment of the disclosure. As with the operation of open loop écraseur 30a (FIGS. 21A through 21I), the targeted organ or tissue 86 is the tendon 220. The open loop écraseur 30e effects the single symmetrical loop configuration 94 of FIGS. 6 and 7.

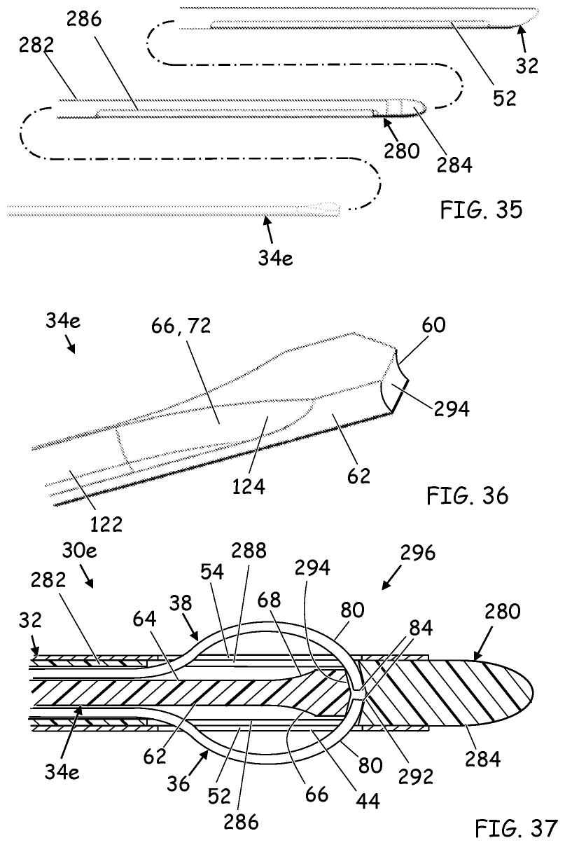

As in FIG. 21A, the trocar 32 may be introduced and passed through the tendon 220, entering at the puncture point 222 and exiting at the exit point 224 along the central axis 42 of the trocar 32. The guide 280 is inserted into the trocar 32 (FIG. 35) so that the alignment head 284 is distal to the lateral openings 52, 54 and the lateral openings 286, 288 are in tangential alignment with the lateral openings 52, 54 (FIG. 38A). The central stem 34e is inserted into the guide 280 (FIG. 35). For embodiments utilizing the deflection portions 66, 68 for routing of the steerable catheters 36, 38, the central stem 34e is positioned to deflect the steerable catheters 36, 38 through the lateral openings 52, 54 near the proximal ends 101 (FIG. 38B).

The steerable catheters 36, 38 are advanced in the distal direction 74 along the lateral sides 62, 64 of the central stem 34e. The distal extremities 84 of the steerable catheters 36, 38 are directed radially outward, away from the central axis 42, through the lateral openings 52, 54 near the proximal ends 101. The initial exodus of the distal extremities 84 may be accomplished by deflection of the steerable catheters 36, 38 with the deflector portions 66, 68 (depicted) or in one or a combination the of the several ways described attendant to FIG. 21D.

Upon exiting the lateral openings 52, 54, advancement of the steerable catheters 36, 38 continues in the distal direction 74 with the distal extremities 84 being steered around the tendon 220 to reenter the lateral openings 52, 54 near the distal ends 103 and proximal to the distal alignment head 284 (FIG. 38C). The central stem 34e is advanced distally so that the steerable catheters 36, 38 are clamped or otherwise captured between the distal face 294 of the central stem 34e and the proximal face 292 of the distal alignment head 284 of the guide 280 (FIG. 38D). During this step, the guide 280 may also be translated proximally to bring the distal alignment head 284 into contact with the steerable catheters 36, 38. With the distal steerable catheters 36, 38 so captured, the distal extremities 84 are in lateral alignment.

The cutting wire assembly 40 is advanced through the steerable catheter 36 so that the distal end 118 exits the distal extremity 84 thereof and enters working channel 76 of the steerable catheter 38 (FIG. 38E). The end portion 118 is further advanced (now in the proximal direction 128) through the working channel 76 of the steerable catheter 38 to complete the écraseur loop 90 within the steerable catheters 36 and 38 (FIG. 38F). For cutting wire assemblies 40 that include the guide wire portion 152, the cutting wire assembly 40 is drawn through the steerable catheter 38 until the cutting target 88 is circumscribed by the cutting wire portion 156. The steerable catheters 36 and 38 are retracted into the trocar 32 so that the distal extremities 84 are drawn into the trocar 32 proximally beyond the lateral openings 52, 54 to fully expose the cutting target 88 to the cutting wire assembly 40 (FIG. 38G). The central stem 34e is retracted in the proximal direction 128 and the cutting wire portion 156 is drawn through the cutting target 88 for severance of the tendon 220 (FIG. 38H). The cutting wire assembly 40, guide 280, and open loop écraseur 30e are withdrawn from the cutting target 88 along the same path the trocar 32 was introduced.

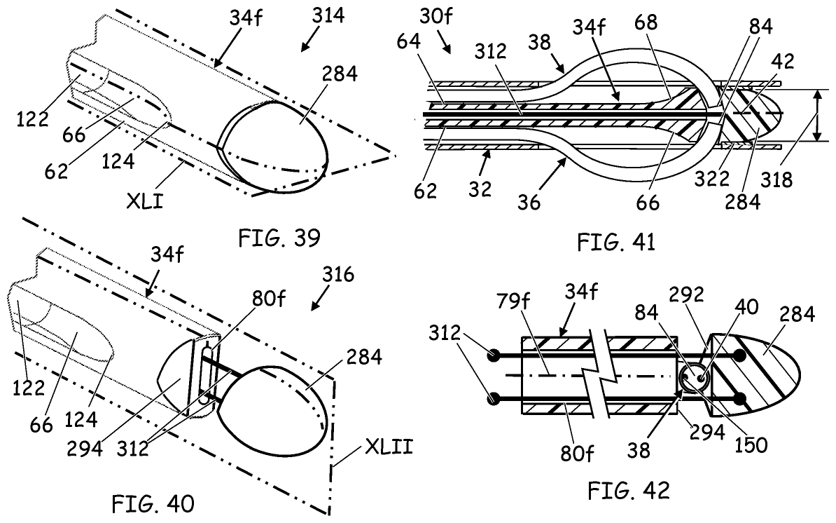

Referring to FIGS. 39 through 42, an open loop écraseur 30f is depicted according to an embodiment of the disclosure. The open loop écraseur 30f includes many of the same components and attributes as the other open loop écraseurs 30 disclosed herein, which are identified by same-labeled reference characters. Like the open loop écraseur 30e, the distal extremities 84 of the steerable catheters 36 and 38 are laterally aligned within the trocar 32 and the cutting wire assembly 40 is routed directly from one steerable catheter 36 to the other steerable catheter 38 without the use of snare assemblies. However, unlike the open loop écraseur 30e, the open loop écraseur 30f does not require the guide 82 to position the distal alignment head 284. Rather, the distal alignment head 284 is coupled to and deployed by a central stem 34f. The central stem 34f may define an internal bypass 80f for manipulation of the distal alignment head 284. Structural and operational aspects for closing the open loop écraseur 30f are described below.

As with the central stem 34e, the central stem 34f includes the distal face 294 that cooperates with the proximal face 292 of the distal alignment head 284. The distal alignment head 284 is coupled to the central stem 34f through the internal bypass 80f, for example with a pair of tethers 312 vertically spaced about a bypass axis 79f. In assembly, the bypass axis 79f is in substantial alignment with the central axis 42 of the trocar 32. In a retracted configuration 314 (FIG. 39), the proximal face 292 of the distal alignment head 284 is in contact with distal face 294 of central stem 34f. In an extended configuration 316 (FIG. 40), the distal alignment head 284 is positioned distally away from and out of physical contact with the central stem 34f. Other mechanisms may be used instead of the tethers 312, for example rods or a flat bar stock with provisions at the distal end for accommodating lateral passage of the cutting wire assembly therethrough.

The trocar 32 is dimensioned to accommodate sliding axial translation of the distal alignment head 284 into the distal end portion 44. In some embodiments, the trocar 32 defines a reduced inner diameter 318 distal to the distal ends 103 of the lateral openings 52, 54, for example by insertion of a bushing 322 (FIG. 41). The reduced inner diameter 318 may be dimensioned for an interference fit with the distal alignment head 284.

Functionally, the tethers 312 may be used to draw the distal alignment head 284 against the central stem 34f, for example prior to insertion into the trocar 32. The vertical spacing of the tethers 312 about the bypass and central axes 79f and 42 enables the end portion 118 to pass freely therebetween without interference from the tethers 312 (FIG. 42), and also enables the cutting wire portion 156 to migrate through the cutting zone 88 without interference from the tethers 312. The dimensioning of the distal alignment head 284 enables free passage through the trocar 32 until reaching the reduced inner diameter 318 distal to the lateral openings 52, 54, wherein the distal alignment head 284 is seated within the reduced inner diameter 318. The tethers 312 can also function to dislodge the distal alignment head 284 from the reduced inner diameter 318 for retrieval or repositioning.

In some embodiments, the cross sections of the steerable catheters 36 and 38 may be different. For example, for the open loop écraseurs 30e and 30f, the steerable catheter 36, which projects the cutting wire assembly 40 the into steerable catheter 38, may include the working channel lumen 186 of FIGS. 15A and 18, whereas the steerable catheter 38 may define the substantially open cross-sectional area 183 of FIG. 15. In this way, the steerable catheter 36 accurately controls the trajectory of the cutting wire assembly 40 into the steerable catheter 38 while the substantially open cross-sectional area 183 of the steerable catheter 38 provides a greater margin for receiving the end portions 118.

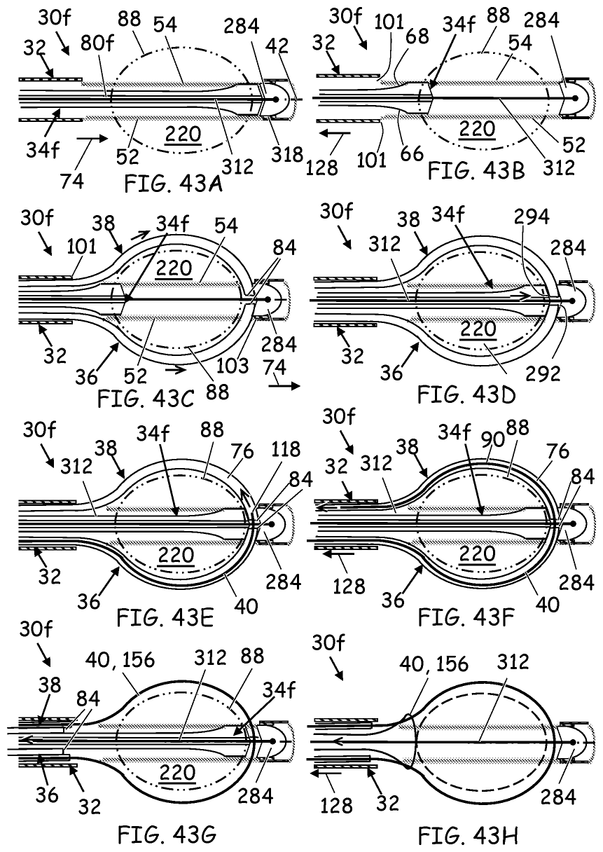

Referring to FIGS. 43A through 43H, operation of the open loop écraseur 30f in a tenotomy procedure is depicted according to an embodiment of the disclosure. As with the operation of open loop écraseur 30a (FIGS. 21A through 21I), the targeted organ or tissue 86 is the tendon 220. The open loop écraseur 30f effects the single symmetrical loop configuration 94 of FIGS. 6 and 7.

As in FIG. 21A, the trocar 32 may be introduced and passed through the tendon 220, entering at the puncture point 222 and exiting at the exit point 224 along the central axis 42 of the trocar 32. The distal alignment head 284 and the central stem 34f are arranged in the retracted configuration 314 and inserted into the trocar 32. The central stem 34f is translated in the distal direction 74 along the central axis 42 so that the distal alignment head 284 is seated within the reduced inner diameter 318 distal to the lateral openings 52 and 54 (FIG. 43A). The central stem 34f is translated in the proximal direction 128, away from the seated distal alignment head 284 (FIG. 43B). For embodiments utilizing the deflection portions 66, 68 for routing of the steerable catheters 36, 38, the central stem 34f is positioned to deflect the steerable catheters 36, 38 through the lateral openings 52, 54 near the proximal ends 101.

The steerable catheters 36, 38 are advanced in the distal direction 74 along the lateral sides 62, 64 of the central stem 34f. The distal extremities 84 of the steerable catheters 36, 38 are directed radially outward, away from the central axis 42, through the lateral openings 52, 54 near the proximal ends 101. The initial exodus of the distal extremities 84 may be accomplished by deflection of the steerable catheters 36, 38 with the deflector portions 66, 68 (depicted) or in one or a combination the of the several ways described attendant to FIG. 21D.

Upon exiting the lateral openings 52, 54, advancement of the steerable catheters 36, 38 continues in the distal direction 74 with the distal extremities 84 being steered around the tendon 220 to reenter the lateral openings 52, 54 near the distal ends 103 and proximal to the distal alignment head 284 (FIG. 43C). The central stem 43f is advanced distally so that the steerable catheters 36, 38 are clamped or otherwise captured between the distal face 294 of the central stem 34f and the proximal face 292 of the distal alignment head 284 (FIG. 43D). During this step, the trocar 32 may also be translated proximally to bring the distal alignment head 284 into contact with the steerable catheters 36, 38. With the distal steerable catheters 36, 38 so captured, the distal extremities 84 are in lateral alignment.

The cutting wire assembly 40 is advanced through the steerable catheter 36 so that the distal end 118 exits the distal extremity 84 thereof and enters working channel 76 of the steerable catheter 38 (FIG. 43E). The end portion 118 is further advanced (now in the proximal direction 128) through the working channel 76 of the steerable catheter 38 to complete the écraseur loop 90 within the steerable catheters 36 and 38 (FIG. 43F). For cutting wire assemblies 40 that include the guide wire portion 152, the cutting wire assembly 40 is drawn through the steerable catheter 38 until the cutting target 88 is circumscribed by the cutting wire portion 156. The steerable catheters 36 and 38 are retracted into the trocar 32 so that the distal extremities 84 are drawn into the trocar 32 proximally beyond the lateral openings 52, 54 to fully expose the cutting target 88 to the cutting wire assembly 40 (FIG. 43G). The central stem 34f is retracted in the proximal direction 128 and the cutting wire portion is 156 drawn through the cutting target 88 for severance of the tendon 220 (FIG. 43H). The cutting wire assembly 40 and open loop écraseur 30f (with distal alignment head 284 and tethers 312 lodged therein) are withdrawn from the cutting target 88 along the same path the trocar 32 was introduced.

Operation of the open loop écraseur 30f without the distal alignment head 284 is also contemplated. After routing the steerable catheters 36, 38 around the tendon 220 and reentering the lateral openings 52, 54, the central stem 34f may be advanced in the distal direction 74 and/or the trocar 32 translated in the proximal direction 128 so that the steerable catheters 36, 38 are registered against the distal ends 103 of the lateral openings 52, 54 (instead of against the distal alignment head 284). By this technique, the lateral alignment of the distal extremities 84 may be sufficient for transferring the cutting wire assembly 40 from steerable catheter 36 to steerable catheter 38.

For the disclosed embodiments, some or all of the components of the disclosed systems and devices may be provided as a kit complete with instructions for use. The instructions are provided on a tangible, non-transitory medium, and may be physically included with the kit such as on a printed document (depicted), compact disc, or flash drive. Non-limiting examples of a tangible, non-transitory medium include a paper document and computer-readable media including compact disc and magnetic storage devices (e.g., hard disk, flash drive, cartridge, floppy drive). The computer-readable media may be local or accessible over the internet. The instructions may be complete on a single medium, or divided among two or more media. For example, some of the instructions may be written on a paper document that instruct the user to access one or more of the steps of the method over the internet, the internet-accessible steps being stored on a computer-readable medium or media. The instructions may embody, in whole or in part, the techniques and methods depicted or described herein, for example using text, photos, videos, or a combination thereof to instruct and guide the user. The instructions may be in the form of written words, figures, photos, video presentations, or a combination thereof to instruct and guide the user.

Herein, the expressions “axial”, “radial”, and “tangential” and their derivatives are in relation to the cylindrical coordinate (r,θ,z) depicted at FIG. 27. The z-coordinate is concentric with the central axis 42 of the trocar 32, but otherwise may be of arbitrary origin. “Axial” refers to directions parallel to the z-coordinate, “radial” refers to directions parallel to the r-coordinate, and “tangential” refers to directions congruent with the θ-coordinate. The expressions “lateral” and “vertical” and their derivatives are in relation to the Cartesian coordinates (x,y,z) depicted at FIG. 28. “Lateral” is the direction that is parallel to the y-coordinate. “Vertical” is the direction parallel to the z-coordinate, and has no relation to the gravity vector. “Distal” and its derivatives refers to a direction that is parallel to the z-coordinate in the positive direction. “Proximal” and its derivatives refers to a direction that is parallel to the z-coordinate in the negative direction.

Each of the additional figures and methods disclosed herein can be used separately, or in conjunction with other features and methods, to provide improved devices and methods for making and using the same. Therefore, combinations of features and methods disclosed herein may not be necessary to practice the disclosure in its broadest sense and are instead disclosed merely to particularly describe representative and preferred embodiments.

Various modifications to the embodiments may be apparent to one of skill in the art upon reading this disclosure. For example, persons of ordinary skill in the relevant arts will recognize that the various features described for the different embodiments can be suitably combined, un-combined, and re-combined with other features, alone, or in different combinations. Likewise, the various features described above should all be regarded as example embodiments, rather than limitations to the scope or spirit of the disclosure.

Persons of ordinary skill in the relevant arts will recognize, in view of this disclosure, that various embodiments can comprise fewer features than illustrated in any individual embodiment described above. The embodiments described herein are not meant to be an exhaustive presentation of the ways in which the various features may be combined. Accordingly, the embodiments are not mutually exclusive combinations of features; rather, the claims can comprise a combination of different individual features selected from different individual embodiments, as understood by persons of ordinary skill in the art.

Any incorporation by reference of documents above is limited such that no subject matter is incorporated that is contrary to the explicit disclosure herein. Any incorporation by reference of documents above is further limited such that no patent claims included in the documents are incorporated by reference herein. Any incorporation by reference of documents above is yet further limited such that any definitions provided in the documents are not incorporated by reference herein unless expressly included herein.

Unless indicated otherwise, references to “embodiment(s)”, “disclosure”, “present disclosure”, “embodiment(s) of the disclosure”, “disclosed embodiment(s)”, and the like contained herein refer to the specification (text, including the claims, and figures) of this patent application that are not admitted prior art.

For purposes of interpreting the claims, it is expressly intended that the provisions of 35 U.S.C. 112(f) are not to be invoked unless the specific terms “means for” or “step for” are recited in the respective claim.

1. An open loop écraseur, comprising:
a trocar that defines and is collinear with a central axis, said trocar including a distal end portion that defines a sharp distal tip for percutaneous insertion, said distal end portion defining a first lateral opening proximate said sharp distal tip;
a central stem dimensioned for a sliding axial translation within said trocar, said central stem including a first lateral side for alignment adjacent said first lateral opening;
a first steerable catheter disposed in said trocar along said first lateral side of said central stem, said first steerable catheter defining a first working channel and including a distal steering section that is extendible radially outward through said first lateral openings, a distal extremity of said steering portion being steerable radially inward toward said first lateral opening;
a cutting wire assembly at least partially disposed in said trocar; and
means for closing an écraseur loop via said first steerable catheter for at least partial circumscription of a cutting target,
wherein said first steerable catheter is retractable within said trocar in a proximal direction to expose said cutting wire assembly for contraction through said cutting target.

2. The open loop écraseur of claim 1, wherein said central stem is retractable within said trocar in said proximal direction to expose said cutting wire assembly for contraction through said cutting target.

3. The open loop écraseur of claim 1, wherein said trocar is metallic.

4. (canceled)

5. The open loop écraseur of claim 1, wherein said first lateral opening defines a first longitudinal axis in a direction parallel to said central axis.

6. The open loop écraseur of claim 1, wherein said central stem includes a first steerable catheter deflector portion for tangential alignment with said first lateral opening, said first steerable catheter deflector portion extending radially outward and in a distal direction for deflection of said first steerable catheter radially outward through said first lateral opening.

7. (canceled)

8. The open loop écraseur of claim 1, wherein said distal steering section of said first steerable catheter includes a steering spine.

9. The open loop écraseur of claim 8, wherein said first steerable catheter includes a steering wire for manipulation of said steering spine.

10. The open loop écraseur of claim 9, wherein said first longitudinal axis and said second longitudinal axis are coplanar with a plane that is radially offset from said central axis.

11. The open loop écraseur of claim 9, wherein said first lateral opening and said second lateral opening are diametrically opposed on said trocar.

12. The open loop écraseur of claim 1, wherein said means for closing said écraseur loop includes at least one snare assembly that defines a snare axis and is axially translatable within an internal bypass defined within said trocar, each of said at least one snare assembly including a snare portion at a distal end thereof for coupling with a first end portion of said cutting wire assembly, said first end portion being disposed in said first working channel of said first steerable catheter, said internal bypass being in fluid communication with said distal end portion of said trocar for translation of said snare portion into said distal end portion of said trocar, said end portion being extendible through said first working channel to exit said distal extremity of said distal steering section of said first steerable catheter for coupling with said snare portion, said snare assembly being retractable in a proximal direction for said at least partial circumscription of said cutting target.

13. The open loop écraseur of claim 12, wherein said first end portion of said cutting wire assembly includes a guide wire at a free end thereof that is attached to a cutting wire.

14. The open loop écraseur of claim 12, wherein said first end portion includes a head portion for secure coupling with said snare portion.

15. The open loop écraseur of claim 12, wherein said snare assembly includes a sleeve selectively translatable over said snare axis for closure of said snare portion.

16.-18. (canceled)

19. The open loop écraseur of claim 12, wherein said snare portion is elastically collapsible.

20. The open loop écraseur of claim 12, wherein said snare portion includes a basket portion.

21. The open loop écraseur of claim 1, wherein said means for closing said écraseur loop includes:
a first snare assembly that is translatable within said first working channel of said first steerable catheter and includes a first snare portion at a distal end thereof for coupling with a first end portion of said cutting wire assembly, said first snare portion being extendible through said first working channel to exit a distal extremity of said distal steering section of said first steerable catheter for positioning said snare portion adjacent and exterior to said first lateral opening of said trocar; and
a first exit port defined at a distal end of said central stem, said first exit port being in fluid communication with an internal bypass defined within said trocar that extends axially and proximally from said first exit port, said first exit port being in fluid communication with said distal end portion of said trocar, said first exit port defining an exit port axis that extends radially outward in a distal direction for deflection of said first end portion through said first lateral opening of said trocar for coupling with said snare portion,
said first end portion of said cutting wire assembly being disposed within said internal bypass and extending through said first exit port, said first snare assembly and said central stem being retractable in a proximal direction for said at least partial circumscription of said cutting target.

22. (canceled)

23. The open loop écraseur of claim 1, wherein said means for closing said écraseur loop includes:
a first snare assembly that is translatable within said first working channel of said first steerable catheter and includes a first snare portion at a distal end thereof for coupling with a first end portion of said cutting wire assembly, said first snare portion being extendible through said first working channel to exit a distal extremity of said distal steering section of said first steerable catheter for positioning said snare portion adjacent and exterior to said first lateral opening of said trocar; and
an internal bypass defined within said trocar that extends axially and is in fluid communication with said distal end portion of said trocar, said first end portion being disposed within said internal bypass and extending into said distal end portion of said trocar and around and proximal to a distal end of said central stem to extend axially and along said first lateral side of said central stem and adjacent said first lateral opening,
wherein translation of said first end portion of said cutting wire assembly in a proximal direction within said internal bypass causes a distal extremity of said first end portion of said cutting wire assembly to deflect radially outward through said first lateral opening for coupling with said first snare portion.

24.-27. (canceled)

28. The open loop écraseur of claim 1, wherein said means for closing said écraseur loop includes:
a snare assembly that is translatable within said first working channel of said first steerable catheter and includes a snare portion at a distal end thereof for coupling with a first end portion of said cutting wire assembly, said snare portion being extendible through said first working channel to exit a distal extremity of said distal steering section of said first steerable catheter for positioning said snare portion adjacent and exterior to said first lateral opening of said trocar; and
a second steerable catheter disposed in said trocar along a second lateral side of said central stem, said second steerable catheter defining a second working channel and including a distal steering section that is extendible radially outward through a second lateral opening defined at said distal end portion of said trocar proximate said sharp distal tip, a distal extremity of said second steerable catheter being steerable radially inward toward said second lateral opening, said first end portion of said cutting wire assembly being disposed in said second working channel of said second steerable catheter, a distal extremity of said first end portion being routed through said first lateral opening and said second lateral opening for coupling with said snare portion,
wherein said snare portion is coupled to said first end portion of said cutting wire assembly and said snare assembly is retracted to draw said cutting wire assembly into said first working channel for said at least partial circumscription of said cutting target.

29.-30. (canceled)

31. The open loop écraseur of claim 28, wherein said central stem includes a first steerable catheter deflector portion and a second steerable catheter deflector portion proximate a distal end of said central stem for tangential alignment with said first lateral opening and said second lateral opening, respectively, said first steerable catheter deflector portion extending radially outward and in a distal direction for deflection of said first steerable catheter radially outward through said first lateral opening, said second steerable catheter deflector portion extending radially outward and in a distal direction for deflection of said first steerable catheter radially outward through said first lateral opening.

32. The open loop écraseur of claim 31, wherein the first and second steerable catheter deflector portions are diametrically opposed on said central stem.

33. (canceled)

34. The open loop écraseur of claim 1, wherein said means for closing said écraseur loop includes:
a first end portion of said cutting wire assembly disposed in said first working channel of said first steerable catheter;
a second steerable catheter disposed in said trocar along a second lateral side of said central stem, said second steerable catheter defining a second working channel and including a distal steering section that is extendible radially outward through a second lateral opening defined at said distal end portion of said trocar proximate said sharp distal tip, a distal extremity of said second steerable catheter being steerable radially inward toward said second lateral opening;
wherein said distal steering section of said first steerable catheter is steerable through said first lateral opening and said distal steering section of said second steerable catheter is steerable through said second lateral opening for lateral alignment of the distal extremities of said first steerable catheter and said second steerable catheter within said distal end portion of said trocar, said first end portion of said cutting wire assembly being extendible from said first working channel into said second working channel to at least partially circumscribe said cutting target with said cutting wire assembly.

35. The open loop écraseur of claim 34, wherein said second working channel at said distal extremity of said second steerable catheter defines a larger open area a than said first working channel at said distal extremity of said first steerable catheter.

36. The open loop écraseur of claim 35, wherein said distal steering section of said first steerable catheter defines at least two lumens and said distal steering section of said second steerable catheter defines a single lumen.

37. The open loop écraseur of claim 34, comprising a guide stem disposed within and dimensioned for sliding axial translation within said trocar, said guide stem including a hollow tubular portion that extends to a distal tip portion, said hollow tubular portion being dimensioned to receive said central stem for sliding axial translation within said hollow tubular portion, said guide stem defining first and second lateral openings configured for tangential alignment with the first and second lateral openings, respectively, of said trocar, said distal tip portion including a proximal face that cooperates with a distal face of said central stem for selective clamping of and said lateral alignment of the distal extremities of said first steerable catheter and said second steerable catheter within said distal end portion of said trocar.

38.-39. (canceled)

40. The open loop écraseur of claim 34, comprising:
a distal alignment head dimensioned for sliding axial translation through said distal end portion of said trocar to proximate a distal end of the first and second lateral openings, of said trocar; and
coupling means for drawing said distal alignment head against a distal face of said central stem to define a retracted configuration,
wherein said distal alignment head is seated within said distal end portion of said trocar proximate said distal end of the first and second lateral openings and said central stem
tether and said central stem is drawn proximally away from said distal alignment head to define an expanded configuration.

41.-47. (canceled)
